(19) United States
(12) Patent Application Publication
(10) Pub. No.: US 2025/0129056 A1
(43) Pub. Date: Apr. 24, 2025

(54) PROCESS FOR PREPARING HISTONE DEMETHYLASE INHIBITORS

(71) Applicant: Celgene Corporation, Summit, NJ (US)

(72) Inventors: Richard M. HEID, JR., Maplewood, NJ (US); Michael J. WILLIAMS, Morris Plains, NJ (US); Maryll E. GEHERTY, Pennington, NJ (US); Jianxin HAN, Green Brook, NJ (US); Jian CHEN, Hillsborough, NJ (US)

(21) Appl. No.: 18/682,370

(22) PCT Filed: Aug. 17, 2022
(86) PCT No.: PCT/US2022/040540

§ 371 Date: Feb. 8, 2024

Related U.S. Application Data
(60) Provisional application No. 63/234,344, filed on Aug. 18, 2021.

(51) Int. Cl. C07D 405/12; B01J 31/02; B01J 31/22; B01J 31/24; C07C 227/18; C07D 311/58

(52) U.S. Cl. CPC C07D 405/12; B01J 31/0241; B01J 31/2291; B01J 31/2409; C07C 227/18; C07D 311/58; B01J 2231/4283; B01J 2531/821; B01J 2531/824

(57) ABSTRACT

Provided herein are methods for preparing 3-({[(4R)-7-{methyl[4-(propan-2-yl)phenyl]amino}-3,4-dihydro-2H-1-benzopyran-4-yl]methyl}amino)pyridine-4-carboxylic acid and novel intermediate compounds for use in preparing histone demethylase inhibitors.

### CROSS-REFERENCE TO RELATED APPLICATIONS

This application claims the benefit of priority of U.S. Provisional Application No. 63/234,344, filed Aug. 18, 2021, which is incorporated by reference herein in its entirety for any purpose.

### FIELD

The present disclosure relates generally to methods of preparing 3-({[(4R)-7-{methyl[4-(propan-2-yl)phenyl]amino}-3,4-dihydro-2H-1-benzopyran-4-yl]methyl}amino)pyridine-4-carboxylic acid and to novel intermediate compounds.

### BACKGROUND

The compound 3-({[(4R)-7-{methyl[4-(propan-2-yl)phenyl]amino}-3,4-dihydro-2H-1-benzopyran-4-yl]methyl}amino)pyridine-4-carboxylic acid (designated herein as Compound 8) is a selective inhibitor of the KDM4 family of histone demethylases (see, e.g., U.S. Pat. No. 9,242,968). The chemical structure of Compound 8 is shown below:

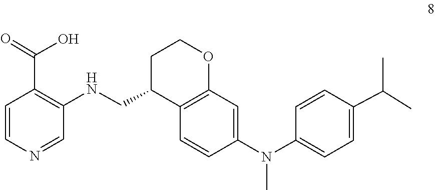 [SMILES: CC(C)c1ccc(N(C)c2ccc3c(c2)OCC[C@H]3CNc2cnccc2C(=O)O)cc1]

This first-in-class epigenetic-modifying compound shows promise for treatment of a variety of cancer types.

To further establish the clinical effectiveness of Compound 8, large quantities of high purity compound are needed. Accordingly, in one aspect, provided herein are methods for preparing 3-({[(4R)-7-{methyl[4-(propan-2-yl)phenyl]amino}-3,4-dihydro-2H-1-benzopyran-4-yl]methyl}amino)pyridine-4-carboxylic acid and salts thereof. Also provided herein are intermediate compounds for use in preparing said compounds.

### SUMMARY

Described herein, in certain embodiments, are methods of preparing 3-({[(4R)-7-{methyl[4-(propan-2-yl)phenyl]amino}-3,4-dihydro-2H-1-benzopyran-4-yl]methyl}amino)pyridine-4-carboxylic acid and salts thereof as histone demethylase inhibitors. Also described herein are intermediate compounds for use in preparing said histone demethylase inhibitors.

The present embodiments can be understood more fully by reference to the detailed description and examples, which are intended to exemplify non-limiting embodiments.

In one aspect, provided herein are methods of preparing Compound 8 or a salt thereof.

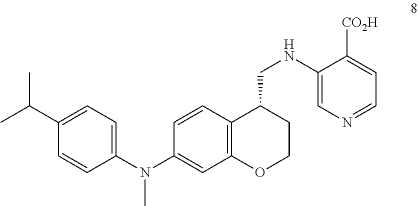 [SMILES: CC(C)c1ccc(N(C)c2ccc3c(c2)OCC[C@H]3CNc2cnccc2C(=O)O)cc1]

In certain aspects, provided herein are methods of preparing Compound 8:

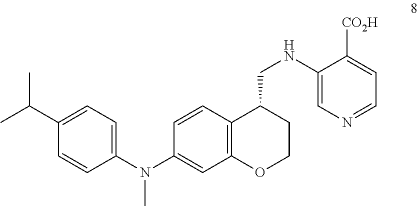 [SMILES: CC(C)c1ccc(N(C)c2ccc3c(c2)OCC[C@H]3CNc2cnccc2C(=O)O)cc1]

or a salt thereof, comprising the following steps:
(a) hydrolyzing Compound 14:

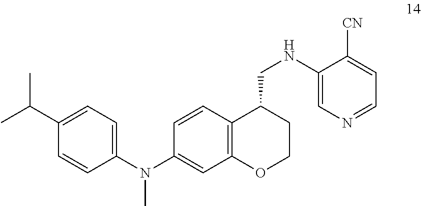 [SMILES: CC(C)c1ccc(N(C)c2ccc3c(c2)OCC[C@H]3CNc2cnccc2C#N)cc1]

or a salt thereof, to form Compound 15:

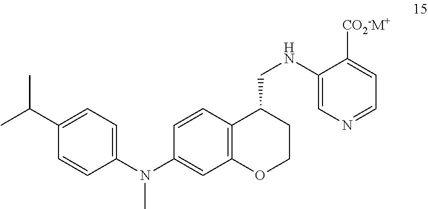

or a solvate thereof;
(b) reacting Compound 15, or a solvate thereof, with acid to form Compound 8; and
(c) optionally converting Compound 8 to a pharmaceutically acceptable salt thereof; and
wherein M⁺ is chosen from alkaline cations and protonated amine bases.

In one aspect, provided herein are novel compounds that are useful as intermediates in the synthesis of Compound 8 or a salt thereof. In certain aspects, provided herein are compounds selected from:

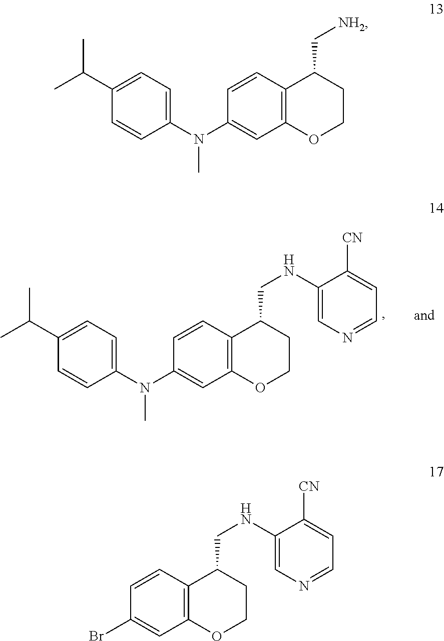 [SMILES: CC(C)c1ccc(N(C)c2ccc3c(c2)OCC[C@H]3CN)cc1 ; CC(C)c1ccc(N(C)c2ccc3c(c2)OCC[C@H]3CNc2cnccc2C#N)cc1 ; N#Cc1ccncc1NC[C@@H]1CCOc2cc(Br)ccc21]

or a salt and/or solvate thereof

### BRIEF DESCRIPTION OF THE DRAWINGS

FIG. 1 shows the results of a pressure screening study that evaluated the effect of pressure in the conversion of Compound 2 to Compound 3 in Alternative Synthesis 1.

FIG. 2 shows the results of a solubility study of Compound 12 from Alternative Synthesis 1 in different solvent systems.

### DETAILED DESCRIPTION

### Definitions

Unless defined otherwise, all technical and scientific terms used herein have the same meaning as is commonly understood by one of skill in the art to which this disclosure belongs. In the following description, certain specific details are set forth in order to provide a thorough understanding of various embodiments of the disclosure. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an”, and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Unless the context requires otherwise, throughout the present specification and claims, the word “comprise” and variations thereof, such as, “comprises” and “comprising” are to be construed in an open, inclusive sense, that is, as “including, but not limited to”.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, ±10%, ±5%, or ±1% of the indicated range, value, or structure, unless otherwise indicated.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, the term “salt” refers to acid or base salts of the compounds disclosed herein. It is understood that “pharmaceutically acceptable salts” are non-toxic. Non-limiting examples of pharmaceutically acceptable salts include acid addition salts and base addition salts.

Pharmaceutically acceptable acid addition salts are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

Pharmaceutically acceptable base addition salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Non-limiting examples of inorganic salts include ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, non-limiting examples of which include ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins, and the like.

“Optional” or “optionally” means that the subsequently described event of circumstances may or may not occur, and that the description includes instances where said event or circumstance occurs and instances in which it does not. For example, “optionally converting Compound X to a salt thereof” means that Compound X may or may not be converted to a salt. In some embodiments, Compound X is converted to a salt, whereas in other embodiments Compound X is not converted to a salt.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Discovery Synthesis

The discovery synthesis described in U.S. Pat. No. 9,242,968 for preparing Compound 8 includes several steps which make it unsuitable for large-scale synthesis including hazardous reaction steps and a C-N coupling reaction which produces polymeric impurities. Thus, there is a need to develop an alternate synthesis for producing Compound 8. In addition, Compound 8 has poor solubility in water and most common organic solvents, and tends to precipitate as an amorphous paste, making filtration at large scale difficult. As such, there is also a need to provide Compound 8 in an alternate form, such as a salt.

The discovery synthesis described in U.S. Pat. No. 9,242,968 for preparing Compound 8 is outlined in Scheme 1.

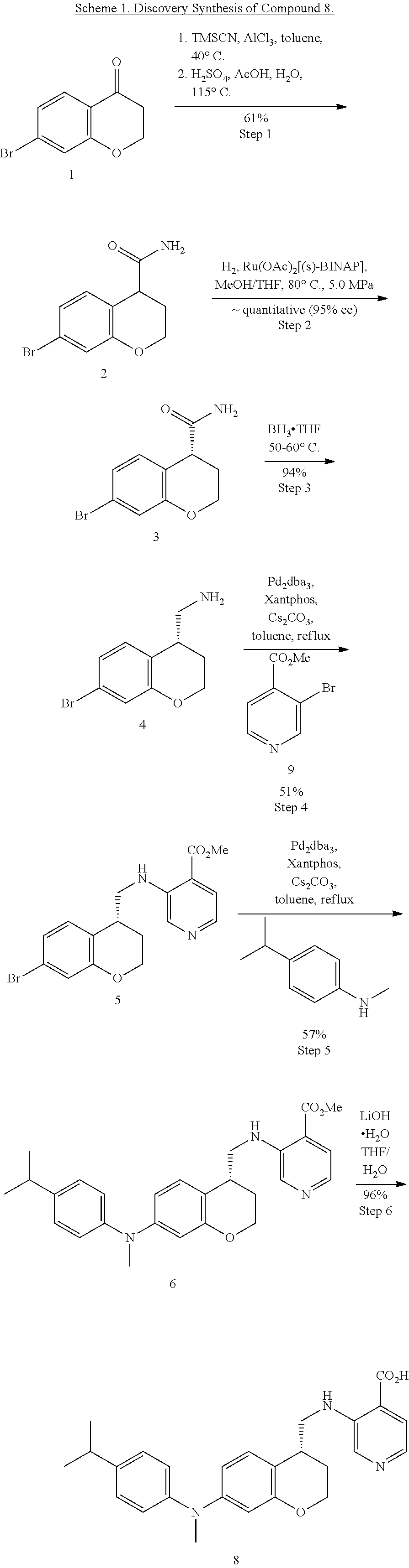

The discovery route begins with the reaction of 7-bromochromanone (Compound 1) with trimethylsilyl cyanide to give the corresponding cyanohydrin, which is then heated with acid to give α,β-unsaturated amide 2. The required chirality is installed via a highly selective (˜95% ee) Ruthenium-catalyzed asymmetric hydrogenation of the double bond to yield Compound 3. The amide is reduced with BH₃ in THF to give the corresponding amine 4. Two consecutive Palladium-catalyzed C-N couplings with Compounds 9 and 10, respectively, afford Compound 6. Hydrolysis of Compound 6 provides Compound 8 in free form.

The discovery synthesis outlined in Scheme 1 yields Compound 8 in 6 linear steps with an overall yield of approximately 16%. However, as described above, the discovery synthesis is not suitable for large-scale production because of several hazardous reaction steps and a C-N coupling reaction which produces polymeric impurities. For example, Step 2 involves high-pressure hydrogenation, Step 3 includes an explosion hazard of the BH₃ THF reaction, and Step 4 has a selectivity issue. In addition, because Compound 8 has poor solubility in most common solvents and tends to precipitate as an amorphous paste, large-scale isolation by filtration is not possible. Therefore, further development of Compound 8 requires (i) an alternate synthetic route which includes elimination of hazardous reactions and a more selective C-N coupling strategy, and (ii) an alternate form of Compound 8 having a more favorable morphology, such as a salt thereof (e.g., a pharmaceutically acceptable salt).

Accordingly, in one aspect, provided herein are alternate synthetic routes that provide 3-({[(4R)-7-{methyl[4-(propan-2-yl)phenyl]amino}-3,4-dihydro-2H-1-benzopyran-4-yl]methyl}amino)pyridine-4-carboxylic acid (Compound 8) as a salt.

### Alternate Synthesis 1

In one aspect, provided herein is a method of preparing Compound 8 or a salt thereof accordingly to the synthetic route outlined in Scheme 2 (“Alternate Synthesis 1”).

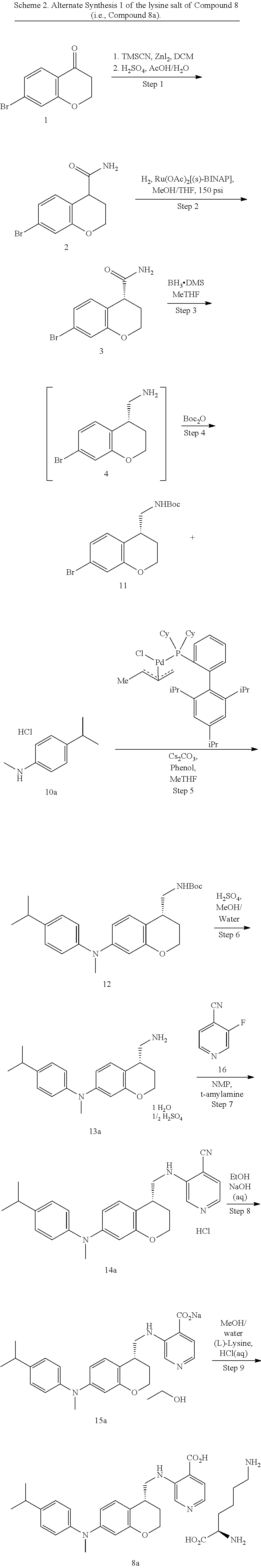

The synthetic strategy of Alternate Synthesis 1 for preparing the lysine salt of Compound 8 as shown in Scheme 2 is centered around the Buchwald coupling reaction between the protected amine 11 and Compound 10a. Other salts can also be made using the Alternate Synthesis 1. This reaction is highly selective and gives the desired coupling product 12 in good yield as the sole product. Alternate Synthesis 1 provides an advantage over the discovery route because only a single C-N coupling step, rather than the two sequential C-N coupling steps (Steps 4 and 5, Scheme 1) are needed, thus providing a more cost-efficient synthesis (reduced cost associated with precious metal catalyst and extra processing associated with heavy metal removal). A significant limitation of the first C-N coupling step of the discovery route (Step 4, Scheme 1) is the formation of polymeric impurities resulting from reaction of the desired product of the reaction, arylbromide 5, with another molecule of amine 4 and so on (see Scheme 3, below).

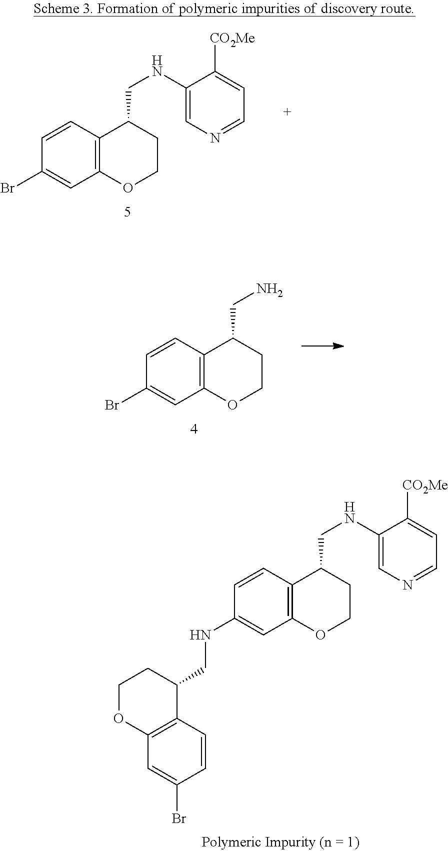 [SMILES: COC(=O)c1ccncc1NC[C@@H]1CCOc2cc(Br)ccc21 ; NC[C@@H]1CCOc2cc(Br)ccc21 ; COC(=O)c1ccncc1NC[C@@H]1CCOc2cc(NC[C@H]3CCOc4cc(Br)ccc43)ccc21]

Polymeric impurities are known to be challenging to remove because they tend to be less soluble than the desired monomer. In addition, as the polymers get bigger, they become extremely challenging to detect. These challenges represent high risk to product purity. A solution to this observed problem is provided in Alternate Synthesis 1.

Another significant drawback of the discovery route for large-scale manufacturing is the high-pressure hydrogenation step (Step 2 of Scheme 1, approximately 725 psi or 5 MPa). Many manufacturing facilities do not have the capability to carry out chemical reactions at such extreme pressure at large scale. A pressure screen showed that there was little to no pressure or temperature effect on the selectivity of the hydrogenation. In all cases, good selectivity was obtained. In addition, the screen showed that a lower hydrogen pressure was sufficient for conversion to product. In this way, Alternate Synthesis 1 eliminates the high-pressure hydrogenation step of the discovery route.

Furthermore, the isolation of high purity product (Compound 3) having reduced residual metal from the hydrogenation reaction of Step 2 proved to be helpful for downstream steps. It was found that activated carbon (e.g., Ecosorb C941) effectively removes residual Ru.

Another drawback to the discovery route is the explosion hazard associated with use of BH₃·THF in the amide reduction of Step 3, Scheme 1. BH₃·THF has a Self-Accelerating Decomposition Temperature (SADT) of 40° C. If this reagent is exposed to adiabatic conditions above 40° C., a self-sustaining exothermic reaction can cause increases in temperature, and exposure of BH₃·THF to temperatures above 60° C. can lead to explosion. The discovery route uses excess BH₃·THF at 50-60° C. To avoid this hazard, the thermally stable BH₃·DMS complex was employed in the amide reduction reaction (Step 3) of Alternate Synthesis 1 (Scheme 2). This complex can be heated at higher temperatures with significantly less risk for a runaway reaction. The BH₃·DMS reaction cleanly produces Compound 4 which could be telescoped directly into the next step. Accordingly, Alternate Synthesis 1 eliminates the explosion hazard associated with the discovery route.

Overall, Alternate Synthesis 1 provides a more efficient route to Compound 8 and salts thereof with superior purity and chiral purity as compared to the discovery route.

Thus, in certain embodiments, disclosed herein is a method of preparing Compound 8:

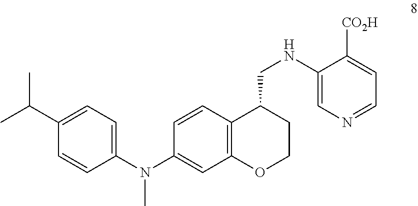 [SMILES: CC(C)c1ccc(N(C)c2ccc3c(c2)OCC[C@H]3CNc2cnccc2C(=O)O)cc1]

or a salt thereof, comprising the following steps:
(a) hydrolyzing Compound 14:

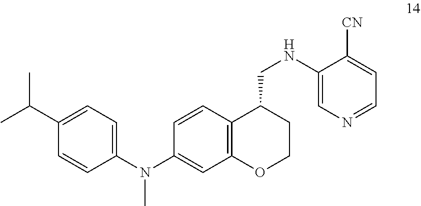 [SMILES: CC(C)c1ccc(N(C)c2ccc3c(c2)OCC[C@H]3CNc2cnccc2C#N)cc1]

or a salt thereof, to form Compound 15:

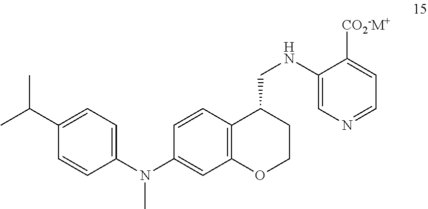

or a solvate thereof;
(b) reacting Compound 15, or a solvate thereof, with acid to form Compound 8; and
(c) optionally converting Compound 8 to a pharmaceutically acceptable salt thereof; and
wherein M⁺ is chosen from alkaline cations and protonated amine bases.

In certain embodiments, M⁺ of Compound 15 is chosen from Na⁺, K⁺, and a protonated dicyclohexylamine. In some embodiments, M⁺ is Na⁺.

In certain embodiments, step (a) is carried out on a salt of Compound 14. In some embodiments, the salt is chosen from an HCl salt, an HBr salt, an HI salt, an H₂SO₄ salt, an H₂PO₄ salt, a methane sulfonic acid salt, a p-toluenesulfonic acid salt, a camphorsulfonic acid salt, an oxalic acid salt, and a benzenesulfonic acid salt. In certain embodiments, the salt is an HCl salt. In some embodiments, the salt of Compound 14 is Compound 14a:

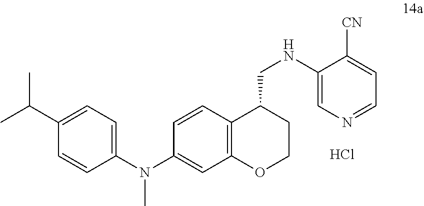 [SMILES: CC(C)c1ccc(N(C)c2ccc3c(c2)OCC[C@H]3CNc2cnccc2C#N)cc1 ; Cl]

In certain embodiments, step (a) is carried out using at least one base. In some embodiments, the at least one base is chosen from an alkaline hydroxide and a dicyclohexylamine. In other embodiments, the alkaline hydroxide is chosen from NaOH and KOH. In some embodiments, the alkaline hydroxide is NaOH.

In certain embodiments, step (a) is carried out using an alcohol as solvent. In some embodiments, the alcohol is chosen from ethanol or methanol. In certain embodiments, the alcohol is ethanol.

In certain embodiments, Compound 15 is formed as a solvate. In some embodiments, Compound 15 is formed as an ethanol or methanol solvate. In certain embodiments, Compound 15 is formed as an ethanol solvate. In some embodiments, the ethanol solvate is Compound 15a:

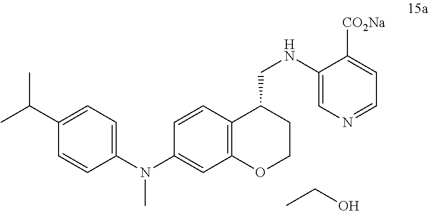

In certain embodiments, the acid in step (b) is chosen from HCl, HBr, HI, H₂SO₄, H₃PO₄, methane sulfonic acid, p-toluenesulfonic acid, camphorsulfonic acid, oxalic acid, and benzenesulfonic acid. In some embodiments, the acid in step (b) is HCl.

In certain embodiments, step (b) is carried out using aqueous alcohol as solvent. In some embodiments, the alcohol is chosen from ethanol or methanol. In certain embodiments, the alcohol is methanol.

In certain embodiments, Compound 8 is reacted with lysine in step (c) to form a lysine salt (Compound 8a).

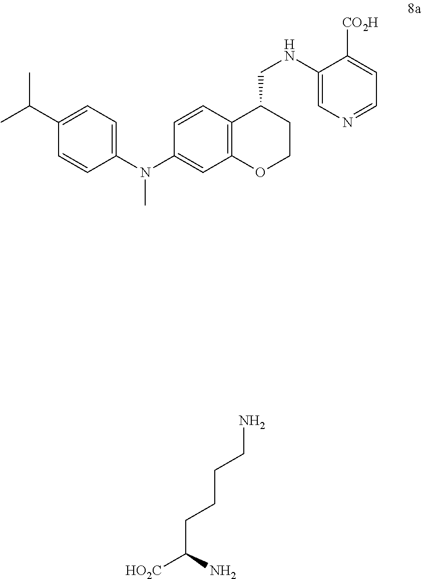 [SMILES: CC(C)c1ccc(N(C)c2ccc3c(c2)OCC[C@H]3CNc2cnccc2C(=O)O)cc1 ; NCCCC[C@@H](N)C(=O)O]

In certain embodiments, Compound 14, or a salt thereof, in step (a) is prepared by the following steps:
(i) reacting Compound 13:

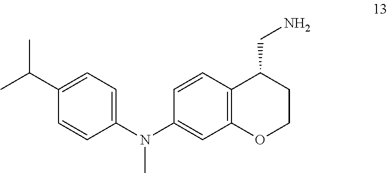 [SMILES: CC(C)c1ccc(N(C)c2ccc3c(c2)OCC[C@H]3CN)cc1]

or a salt and/or solvent thereof,
with Compound 16:

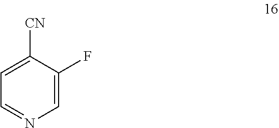

to form Compound 14; and
(ii) optionally converting Compound 14 into a salt thereof.

In certain embodiments, step (i) is carried out using at least one polar aprotic solvent. In some embodiments, the at least one polar aprotic solvent is chosen from N-methyl-2-pyrrolidone (NMP), 2-methyl tetrahydrofuran (2-MeTHF), DMF, DMSO, THF, DMAc, N-methylimidazole, acetonitrile, dimethoxyethane, and 1,4-dioxane. In certain embodiments, the at least one polar aprotic solvent is chosen from N-methyl-2-pyrrolidone (NMP) and 2-methyl tetrahydrofuran (2-MeTHF). In some embodiments, the at least one polar aprotic solvent is N-methyl-2-pyrrolidone (NMP).

In certain embodiments, step (i) is carried out using a base. In some embodiments, the base is chosen from t-amylamine, Cs₂CO₃, pyridine, 1,8-Diazabicyclo[5.4.0]undec-7-ene (DBU), N-methylimidazole (NMI), Et₃N, 1,4-diazabicyclo[2.2.2]octane (Dabco), Borate Buffer, DBU, TMG, NaOTMS, and KHMDS. In some embodiments, the base is chosen from t-amylamine, diisopropylethylamine, tert-butylamine, DBU and TMG. In certain embodiments, the base is t-amylamine.

In certain embodiments, step (i) is carried out at a temperature of about 60-100° C. In some embodiments, the temperature is of about 70-90° C. In certain embodiments, the temperature is of about 70-80° C.

In certain embodiments, Compound 14 is reacted with an acid in step (ii) to form a salt. In some embodiments, the acid in step (ii) is chosen from HCl, HBr, HI, H₂SO₄, H₃PO₄, methane sulfonic acid, p-toluenesulfonic acid, camphorsulfonic acid, oxalic acid, and benzenesulfonic acid. In certain embodiments, the acid in step (ii) is HCl.

In certain embodiments, Compound 13, or a salt and/or solvate thereof, is prepared by deprotecting Compound 12:

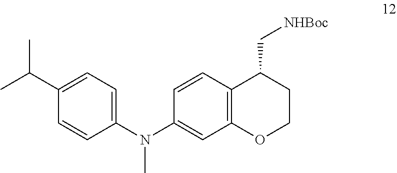 [SMILES: CC(C)c1ccc(N(C)c2ccc3c(c2)OCC[C@H]3CNC(=O)OC(C)(C)C)cc1]

under acidic conditions to form Compound 13, or salt and/or solvate thereof.

In certain embodiments, the acid conditions comprise H₂SO₄, HCl, HBr, and/or benzenesulfonic acid. In certain embodiments, the acidic conditions comprise H₂SO₄. In certain embodiments, the acidic conditions comprise H₂SO₄ in aqueous alcohol. In some embodiments, the alcohol is methanol or ethanol. In certain embodiments, the alcohol is methanol.

In certain embodiments, the deprotecting of Compound 12 to form Compound 13, or a salt and/or solvate thereof, is carried out at a temperature of about 35-55° C. In some embodiments, the deprotection is carried out at a temperature of about 35-45° C.

In certain embodiments, Compound 12 is prepared by reacting Compound 11:

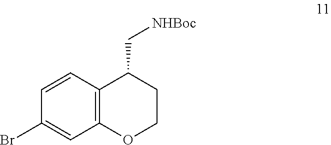 [SMILES: CC(C)(C)OC(=O)NC[C@@H]1CCOc2cc(Br)ccc21]

with Compound 10a:

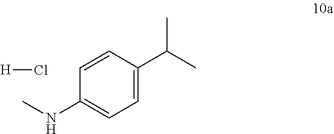

using a Palladium catalyst and phenol to form Compound 12.

In certain embodiments, the Palladium catalyst is chosen from Xphos Pd(crotyl)Cl, RuPhos Pd G2, XPhos Pd G2, BrettPhos Pd G3, CPhos Pd G3, DavePhos Pd G3, P(tBu)3 Pd G2, JosiPhos Pd G3, MorDalPhos Pd G3, BINAP Pd G3, SPhos Pd G2, SPhos Pd G2, tBuXPhos Pd G3, XantPhos Pd G3, and XPhos Pd G3. In some embodiments, the Palladium catalyst is chosen from:

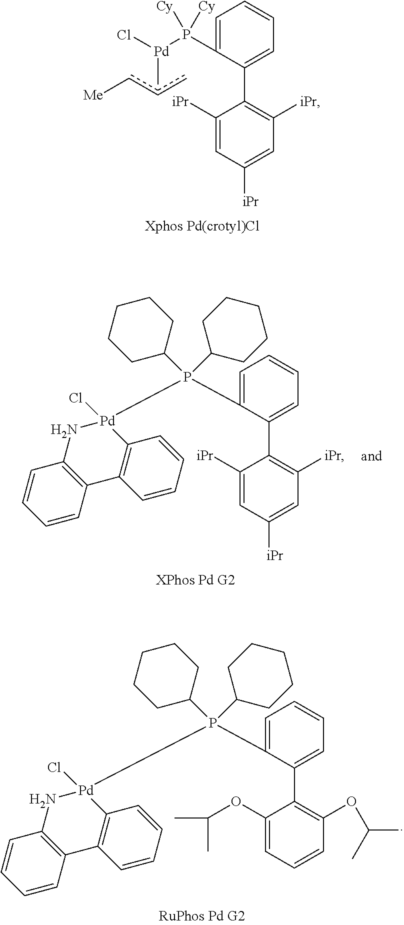

In certain embodiments, the Palladium catalyst is

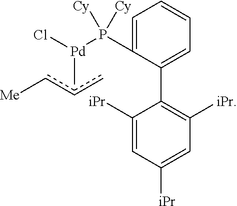

In certain embodiments, the Palladium catalyst is used in an amount of about 1-3 mole %. In some embodiments, the Palladium catalyst is used in an amount of about 2 mole %.

In certain embodiments, the reaction of Compound 11 with Compound 10a is carried out using a solvent chosen from THF and 2-methyltetrahydrofuran. In some embodiments, the solvent is 2-methyltetrahydrofuran.

In certain embodiments, the reaction of Compound 11 with Compound 10a is carried out at a temperature of about 70-90° C. In some embodiments, the temperature is about 80° C. In certain embodiments, the temperature is about 75° C.

In certain embodiments, the reaction of Compound 11 with Compound 10a is carried out in the presence of a base. In some embodiments, the base is chosen from NaOPh and Cs₂CO₃. In certain embodiments, the base is Cs₂CO₃. In some embodiments, the Cs₂CO₃ is milled.

In certain embodiments, Compound 11 is prepared by reacting Compound 4:

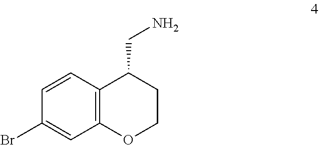 [SMILES: NC[C@@H]1CCOc2cc(Br)ccc21]

with Boc₂O to form Compound 11.

In certain embodiments, Compound 4 is prepared by reacting Compound 3:

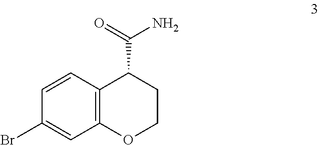 [SMILES: NC(=O)[C@@H]1CCOc2cc(Br)ccc21]

with BH₃·DMS to form Compound 4.

In certain embodiments, the reaction of Compound 3 with BH₃·DMS is carried out using a solvent chosen from toluene, tetrahydrofuran, and 2-methyltetrahydrofuran. In some embodiments, the solvent is 2-methyltetrahydrofuran.

In certain embodiments, the reaction of Compound 3 with BH₃·DMS is carried out at a temperature of about 55-65° C. In certain embodiments, the temperature is about 70° C.

In certain embodiments, Compound 3 is prepared by hydrogenating Compound 2:

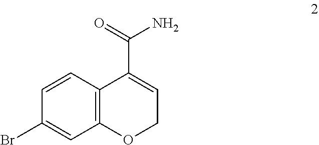 [SMILES: NC(=O)C1=CCOc2cc(Br)ccc21]

using a Ruthenium catalyst to form Compound 3. In some embodiments, the Ruthenium catalyst is chosen from (s)-RuCl[(p-cymene)(DM-SEGPHOS®)]Cl, (s)-RuCl[(p-cymene)(DTBM-SEGPHOS®)]Cl, (S)-RuCl[(p-cymene)(BINAP)]Cl, (s)-RuCl[(p-cymene)(T-BINAP)]Cl, (s)-RuCl[p-cymene)(H8-BINAP)]Cl, (s)-RuCl[(p-cymene)(SEGPHOS®)]Cl and Ru(OAc)₂[(s)-BINAP]. In certain embodiments, the Ruthenium catalyst is Ru(OAc)₂[(s)-BINAP].

In certain embodiments, the hydrogenating is carried out using methanol, tetrahydrofuran, or a combination thereof as solvent. In some embodiments, the hydrogenating is carried out using a combination of methanol and tetrahydrofuran as solvent.

In certain embodiments, the hydrogenating is carried out at a temperature of about 30-50° C. In some embodiments, the temperature is about 30-45° C. In certain embodiments, the temperature is of about 35° C.

In certain embodiments, the hydrogenating is carried out under high pressure. In some embodiments, the hydrogenating is carried out using a pressure of about 60-200 psi. In certain embodiments, the pressure is about 75-200 psi. In some embodiments, the pressure is about 100-150 psi. In certain embodiments, the pressure is about 150 psi.

In certain embodiments, Compound 2 is prepared by reacting Compound 1:

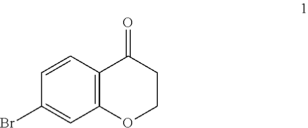 [SMILES: O=C1CCOc2cc(Br)ccc21]

with (i) trimethylsilyl cyanide and (ii) acid to form Compound 2. In certain embodiments, the reaction of Compound 1 with trimethylsilyl cyanide in (i) is carried out using ZnI₂ or ZnCl₂. In some embodiments, the reaction of Compound 1 with trimethylsilyl cyanide in (i) is carried out using ZnCl₂. In certain embodiments, the reaction of Compound 1 with trimethylsilyl cyanide in (i) is carried out using ZnI₂.

In certain embodiments, the reaction of Compound 1 with trimethylsilyl cyanide in (i) is carried out using a solvent chosen from toluene, dichloromethane, and dichloromethane. In some embodiments, the solvent is dichloromethane

In certain embodiments, in the preparation of Compound 2, the acid in (ii) is sulfuric acid, acetic acid, or a combination thereof. In some embodiments, the acid in (ii) is sulfuric acid. In certain embodiments, the acid in (ii) is acetic acid. In some embodiments, the acid in (ii) is a combination of sulfuric acid and acetic acid.

In certain embodiments, in the preparation of Compound 2, the reaction with acid in (ii) is carried out at a temperature of about 50-100° C. In some embodiments, the temperature is of about 50-90° C. In some embodiments, the temperature is of about 50-80° C. In certain embodiments, the temperature is about 70-80° C.

### Alternate Synthesis 2

In another aspect, provided herein is a method of preparing the Compound 8 or a salt thereof accordingly to the synthetic route outlined in Scheme 4 (“Alternate Synthesis 2”).

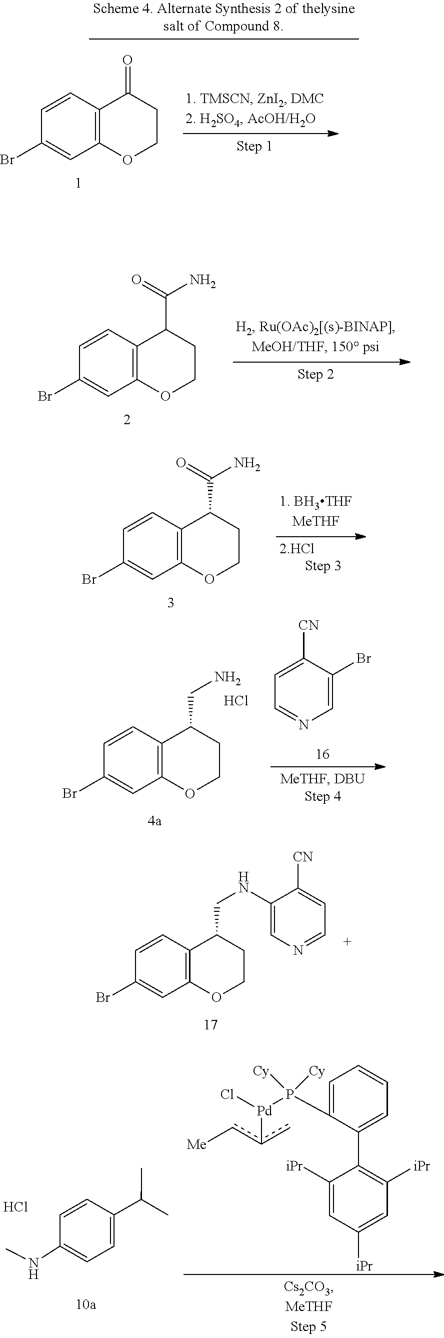

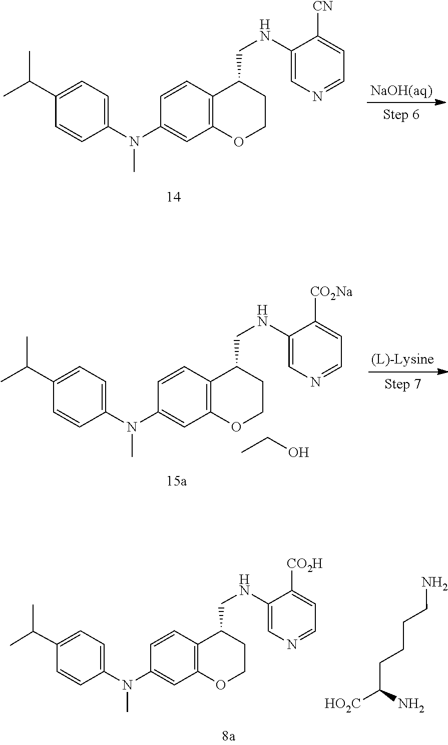

The synthetic strategy for preparing the lysine salt of Compound 8 as shown in Scheme 4 is a modification of the discovery route (Scheme 1) which uses the same bond forming sequence. Other salts can also be made using the Alternate Synthesis 2. Alternate Synthesis 2 replaces the unselective Buchwald coupling reaction (Step 4, Scheme 1) with a selective S_{N}Ar reaction of Compound 4a with 4-cyano-3-fluoropyridine (Compound 16) to give Compound 17. The coupling of Compound 17 with Compound 10a provides Compound 14, which is also an intermediate in Alternate Synthesis 1.

Alternate Synthesis 2 requires only a single C-N coupling step, in contrast to the two consecutive C-N coupling steps needed in the discovery route (Steps 4 and 5, Scheme 1). Therefore, Alternate Synthesis 2 is a more cost-efficient synthesis due to reduced cost associated with precious metal catalyst and extra processing associated with heavy metal removal. In addition, as described above for Alternate Synthesis 1, a significant limitation of the first C-N coupling step of the discovery route (Step 4, Scheme 1) is the formation of polymeric impurities resulting from reaction of the desired product of the reaction (Compound 5) with another molecule of amine 4 and so on (Scheme 3). The difficulties associated with identifying and removing the polymeric impurities impact the product purity afforded by the discovery route. This problem can be solved by using Alternate Synthesis 2.

Alternate Synthesis 2 also eliminates the high-pressure hydrogenation step of the discovery route (Step 2 of Scheme 1, approximately 725 psi or 5 MPa). A hydrogen pressure of 150 psi was found to be sufficient for the hydrogenation.

Alternate Synthesis 2 utilizes the thermally stable BH₃·DMS complex in the amide reduction reaction (Step 3, Scheme 4), in contrast to the potentially explosive BH₃·THF reagent used in the discovery route (Step 3, Scheme 1). As described above, BH₃·DMS can be heated at higher temperatures with significantly less risk for a runaway reaction. Accordingly, Alternate Synthesis 2 eliminates the explosion hazard associated with the discovery route.

Overall, Alternate Synthesis 2 provides a more efficient route to Compound 8 and salts thereof with superior purity and chiral purity as compared to the discovery route.

Thus, in certain embodiments, disclosed herein is a method of preparing Compound 8:

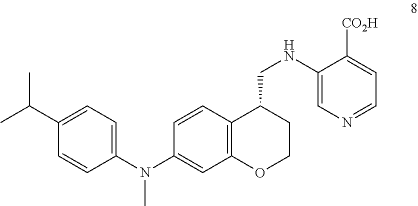 [SMILES: CC(C)c1ccc(N(C)c2ccc3c(c2)OCC[C@H]3CNc2cnccc2C(=O)O)cc1]

or a salt thereof, comprising the following steps:
(a) hydrolyzing Compound 14:

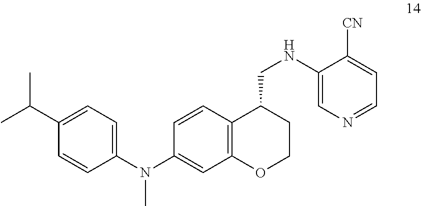 [SMILES: CC(C)c1ccc(N(C)c2ccc3c(c2)OCC[C@H]3CNc2cnccc2C#N)cc1]

or a salt thereof, to form Compound 15:

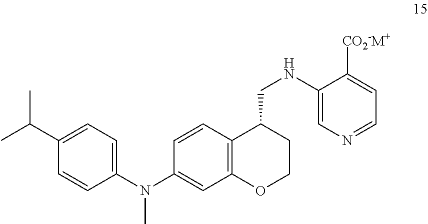

or a solvate thereof;
(b) reacting Compound 15, or a solvate thereof, with acid to form Compound 8; and
(c) optionally converting Compound 8 to a pharmaceutically acceptable salt thereof; and
wherein M⁺ is chosen from alkaline cations and protonated amine bases.

In certain embodiments, M⁺ of Compound 15 is chosen from Na⁺, K⁺, and a protonated dicyclohexylamine. In some embodiments, M⁺ is Na⁺.

In certain embodiments, step (a) is carried out on a salt of Compound 14. In some embodiments, the salt is chosen from an HCl salt, an HBr salt, an HI salt, an H₂SO₄ salt, an H₃PO₄ salt, a methane sulfonic acid salt, a p-toluenesulfonic acid salt, a camphorsulfonic acid salt, an oxalic acid salt, and a benzenesulfonic acid salt. In certain embodiments, the salt is an HCl salt. In some embodiments, the salt of Compound 14 is Compound 14a:

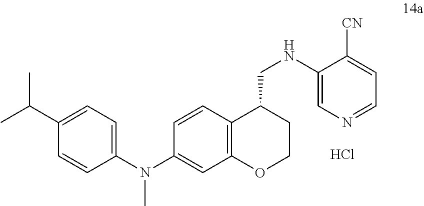 [SMILES: CC(C)c1ccc(N(C)c2ccc3c(c2)OCC[C@H]3CNc2cnccc2C#N)cc1 ; Cl]

In certain embodiments, step (a) is carried out using at least one base. In some embodiments, the at least one base is chosen from an alkaline hydroxide and a dicyclohexylamine. In other embodiments, the alkaline hydroxide is chosen from NaOH and KOH. In some embodiments, the alkaline hydroxide is NaOH.

In certain embodiments, step (a) is carried out using an alcohol as solvent. In some embodiments, the alcohol is chosen from ethanol or methanol. In certain embodiments, the alcohol is ethanol.

In certain embodiments, Compound 15 is formed as a solvate. In some embodiments, Compound 15 is formed as an ethanol or methanol solvate. In certain embodiments, Compound 15 is formed as an ethanol solvate. In some embodiments, the ethanol solvate is Compound 15a:

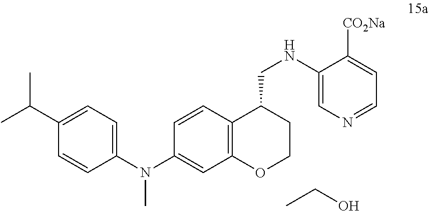

In certain embodiments, the acid in step (b) is chosen from HCl, HBr, HI, H₂SO₄, H₃PO₄, methane sulfonic acid, p-toluenesulfonic acid, camphorsulfonic acid, oxalic acid, and benzenesulfonic acid. In some embodiments, the acid in step (b) is HCl.

In certain embodiments, step (b) is carried out using aqueous alcohol as solvent. In some embodiments, the alcohol is chosen from ethanol or methanol. In certain embodiments, the alcohol is methanol.

In certain embodiments, Compound 8 is reacted with lysine in step (c) to form a lysine salt (Compound 8a).

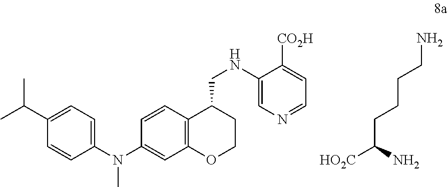 [SMILES: CC(C)c1ccc(N(C)c2ccc3c(c2)OCC[C@H]3CNc2cnccc2C(=O)O)cc1 ; NCCCC[C@@H](N)C(=O)O]

In certain embodiments, Compound 14, or a salt thereof, is prepared by reacting Compound 17:

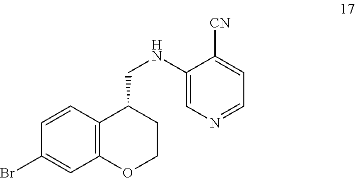 [SMILES: N#Cc1ccncc1NC[C@@H]1CCOc2cc(Br)ccc21]

with Compound 10a:

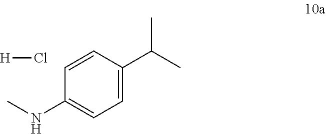

using a Palladium catalyst and phenol to form Compound 14, or a salt thereof.

In certain embodiments, the Palladium catalyst is chosen from Xphos Pd(crotyl)Cl, RuPhos Pd G2, XPhos Pd G2, BrettPhos Pd G3, CPhos Pd G3, DavePhos Pd G3, P(tBu)3 Pd G2, JosiPhos Pd G3, MorDalPhos Pd G3, BINAP Pd G3, SPhos Pd G2, SPhos Pd G2, tBuXPhos Pd G3, XantPhos Pd G3, and XPhos Pd G3. In some embodiments, the Palladium catalyst is chosen from:

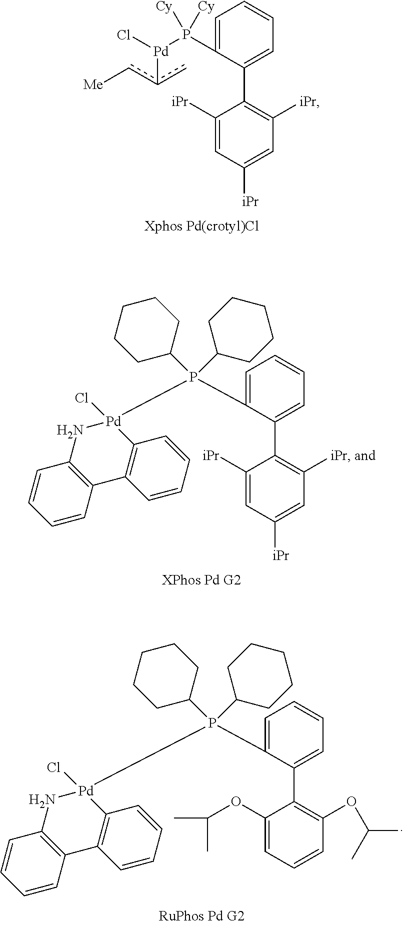

In certain embodiments, the Palladium catalyst is

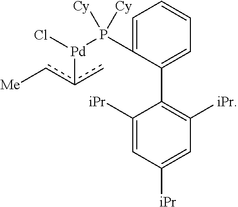

In certain embodiments, the Palladium catalyst is used in an amount of at least about 2 mole %. In some embodiments, the Palladium catalyst is used in an amount of about 3 mole %.

In certain embodiments, the reaction of Compound 17 with Compound 10a is carried out using a solvent chosen from THF, toluene, dioxane, and 2-methyltetrahydrofuran, with and without water. In some embodiments, the solvent is 2-methyltetrahydrofuran.

In certain embodiments, the reaction of Compound 17 with Compound 10a is carried out at a temperature of about 70-90° C. In some embodiments, the temperature is about 75-80° C. In certain embodiments, the temperature is about 80° C.

In certain embodiments, the reaction of Compound 17 with Compound 10a is carried out in the presence of a base. In some embodiments, the base is chosen from Et₃N, DIPEA, DBU, and Cs₂CO₃. In certain embodiments, the base is Cs₂CO₃. In some embodiments, the Cs₂CO₃ is milled.

In certain embodiments, Compound 17 is prepared by reacting Compound 4a:

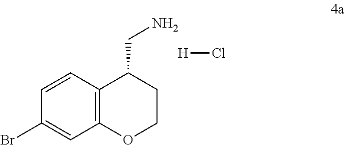

with Compound 16:

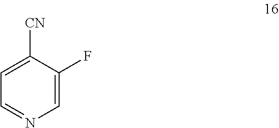

to form Compound 17.

In certain embodiments, the reaction of Compound 4a with Compound 16 is carried out using a catalyst chosen from TMG, t-butylamine, tert-amyl amine, and DBU. In some embodiments, the catalyst is DBU.

In certain embodiments, the reaction of Compound 4a with Compound 16 is carried out using a solvent chosen from DMSO, DMF, DMAc, NMP, N-methylimidazole, acetonitrile, dimethoxyethane, 1,4-dioxane, and 2-methyltetrahydrofuran. In some embodiments, the solvent is 2-methyltetrahydrofuran.

In certain embodiments, the reaction of Compound 4a with Compound 16 is carried out at a temperature of about 30-70° C. In some embodiments, the temperature is about 40-60° C. In certain embodiments, the temperature is of about 50° C.

In certain embodiments, Compound 4a is prepared by reacting Compound 3:

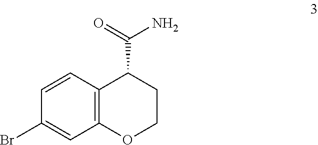 [SMILES: NC(=O)[C@@H]1CCOc2cc(Br)ccc21]

with BH₃·DMS and HCl to form Compound 4a.

In certain embodiments, the reaction of Compound 3 with BH₃·DMS and HCl is carried out using a solvent chosen from toluene, tetrahydrofuran, and 2-methyltetrahydrofuran. In some embodiments, the solvent is 2-methyltetrahydrofuran

In certain embodiments, the reaction of Compound 3 with BH₃·DMS and HCl is carried out at a temperature of about 40-90° C. In some embodiments, the temperature is about 50-80° C. In some embodiments, the temperature is about 60-70° C. In certain embodiments, the temperature is of about 70° C.

In certain embodiments Compound 3 is prepared by hydrogenating Compound 2:

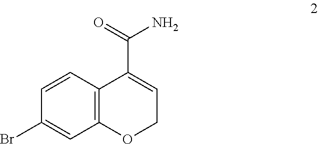 [SMILES: NC(=O)C1=CCOc2cc(Br)ccc21]

using a Ruthenium catalyst to form Compound 3. In certain embodiments, the Ruthenium catalyst is chosen from (s)-RuCl[(p-cymene)(DM-SEGPHOS®)]Cl, (s)-RuCl[(p-cymene)(DTBM-SEGPHOS®)]Cl, (S)-RuCl[(p-cymene)(BINAP)]Cl, (s)-RuCl[(p-cymene)(T-BINAP)]Cl, (s)-RuCl[p-cymene)(H8-BINAP)]Cl, (s)-RuCl[(p-cymene)(SEGPHOS®)]Cl and Ru(OAc)₂[(s)-BINAP]. In some embodiments, the Ruthenium catalyst is Ru(OAc)₂[(s)-BINAP].

In certain embodiments, the hydrogenating is carried out using methanol, tetrahydrofuran, or a combination thereof as solvent. In some embodiments, the hydrogenating is carried out using a combination of methanol and tetrahydrofuran as solvent.

In certain embodiments, the hydrogenating is carried out at a temperature of about 30-50° C. In some embodiments, the temperature is about 30-40° C. In certain embodiments, the temperature is of about 35° C.

In certain embodiments, the hydrogenating is carried out under high pressure. In some embodiments, the hydrogenating is carried out using a pressure of about 60-200 psi. In certain embodiments, the pressure is about 75-200 psi. In some embodiments, the pressure is about 100-150 psi. In certain embodiments, the pressure is about 150 psi.

In certain embodiments, Compound 2 is prepared by reacting Compound 1:

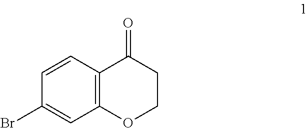 [SMILES: O=C1CCOc2cc(Br)ccc21]

with (i) trimethylsilyl cyanide and (ii) acid to form Compound 2. In certain embodiments, the reaction of Compound 1 with trimethylsilyl cyanide in (i) is carried out using ZnI₂ or ZnCl₂. In some embodiments, the reaction of Compound 1 with trimethylsilyl cyanide in (i) is carried out using ZnCl₂. In certain embodiments, the reaction of Compound 1 with trimethylsilyl cyanide in (i) is carried out using ZnI₂.

In certain embodiments, the reaction of Compound 1 with trimethylsilyl cyanide in (i) is carried out using a solvent chosen from toluene, dichloromethane, and dichloromethane. In some embodiments, the solvent is dichloromethane

In certain embodiments, in the preparation of Compound 2, the acid in (ii) is sulfuric acid, acetic acid, or a combination thereof. In some embodiments, the acid in (ii) is sulfuric acid. In certain embodiments, the acid in (ii) is acetic acid. In some embodiments, the acid in (ii) is a combination of sulfuric acid and acetic acid.

In certain embodiments, in the preparation of Compound 2, the reaction with acid in (ii) is carried out at a temperature of about 50-100° C. In some embodiments, the temperature is of about 50-90° C. In some embodiments, the temperature is of about 50-80° C. In certain embodiments, the temperature is about 70-80° C.

### Intermediate Compounds

In one aspect, provided herein are novel compounds that are useful as intermediates in the synthesis of Compound 8 or a salt thereof. In certain embodiments, the compound is chosen from compound selected from:

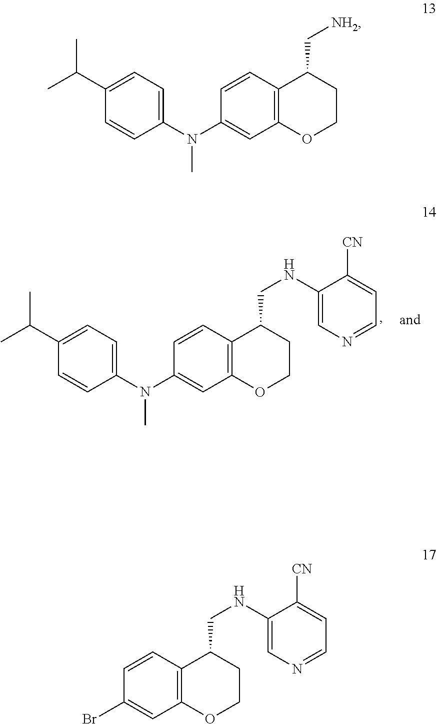 [SMILES: CC(C)c1ccc(N(C)c2ccc3c(c2)OCC[C@H]3CN)cc1 ; CC(C)c1ccc(N(C)c2ccc3c(c2)OCC[C@H]3CNc2cnccc2C#N)cc1 ; N#Cc1ccncc1NC[C@@H]1CCOc2cc(Br)ccc21]

or a salt and/or solvate thereof.

In certain embodiments, the compound is Compound 13 or salt and/or solvate thereof. In some embodiments, the compound is a salt of Compound 13. In certain embodiments, the compound is a solvate of Compound 13. In some embodiments, the compound is a solvate-salt of Compound 13. In certain embodiments, the compound is:

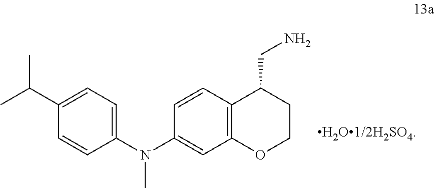 [SMILES: CC(C)c1ccc(N(C)c2ccc3c(c2)OCC[C@H]3CN)cc1]

In certain embodiments, the compound is Compound 14 or a salt and/or solvate thereof. In some embodiments, the compound is a salt of Compound 14. In certain embodiments, the compound is:

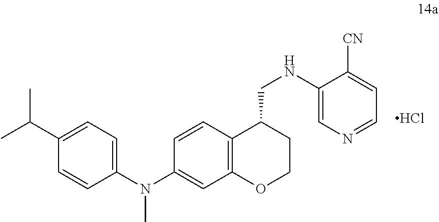 [SMILES: CC(C)c1ccc(N(C)c2ccc3c(c2)OCC[C@H]3CNc2cnccc2C#N)cc1 ; Cl]

In certain embodiments, the compound is Compound 17 or a salt and/or solvate thereof. In some embodiments, the compound is a salt of Compound 17. In certain embodiments, the compound is a solvate thereof.

### Examples

These examples are provided for illustrative purposes only and not to limit the scope of the claims provided herein.

¹H and ¹³C nuclear magnetic resonance spectra (NMR) were obtained on a Bruker 300 MHz, 400 MHz or 500 MHz spectrometer and values reported in ppm (δ) referenced against residual CHCl₃, CD₃SO-CD₃, etc. Spin-spin coupling constants are described as singlet (s), doublet (d), triplet (t), quartet (q), quintet (quint), broad (br) or multiplet (m), with coupling constants (J) in Hz. Mass spectra were obtained using an Agilent 6230B time-of-flight (ToF) mass spectrometer. Accurate mass analyses were performed in ESI positive and negative ion mode using CAPSO as an internal calibrant.

Abbreviations used:

| | |
|---|---|
| AcOH or HOAc | Acetic acid |
| BINAP Pd G3 | 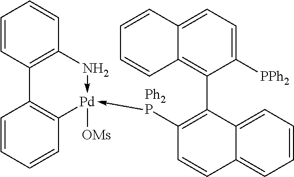 |
| Boc₂O | Di-tert-butyl dicarbonate |
| BrettPhos Pd G3 | 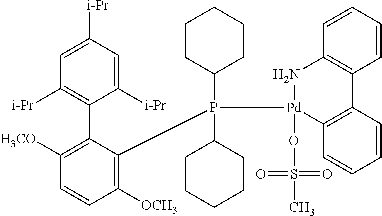 |
| BSA | Benzenesulfonic acid |
| CPhos Pd G3 | 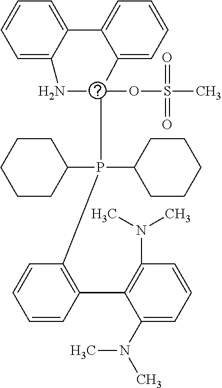 |
| CPMe | Cyclopentyl methyl ether |
| Dabco | 1,4-diazabicyclo[2.2.2]octane |
| DavePhos Pd G3 | 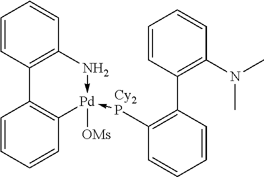 |
| DBU | 1,8-Diazabicyclo[5.4.0]undec-7-ene |
| DCM | Dichloromethane |
| DIPEA | N,N-Diisopropylethylamine |
| DMAc | Dimethylacetamide |
| DMS | Dimethyl sulfide |
| DP:IS | Desired product area versus internal standard area |
| ee | Enantiomeric excess |
| HTE | High-Throughput Experimentation |
| IPA | Isopropyl acetate |
| IPAc | Isopropyl acetate |
| JosiPhos Pd G3 | 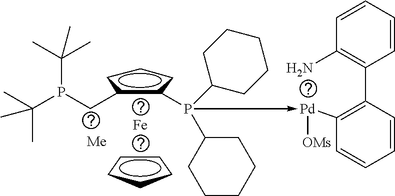 |
| KHMDS | Potassium bis(trimethylsilyl)amide |
| MBTE or MTBE | Methyl tert-butyl ether |
| MEK | Methyl ethyl ketone |
| MeTHF or 2-MeTHF | 2-Methyltetrahydrofuran |
| MIBK | Methyl isobutyl ketone |
| MorDalPhos Pd G3 | 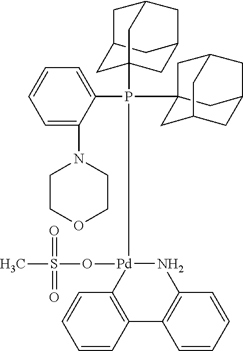 |
| NaOTMS | Sodium trimethylsilanolate |
| N/D | Not determined |
| NLT | Not Less Than |
| NMI | N-methylimidazole |
| NMP | N-methyl-2-pyrrolidine |
| NMT | No More Than |
| psi | Pounds per square inch |
| P(tBu)3 Pd G2 | 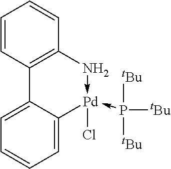 |
| ROI | Residue on Ignition |
| RuPhos Pd G2 | 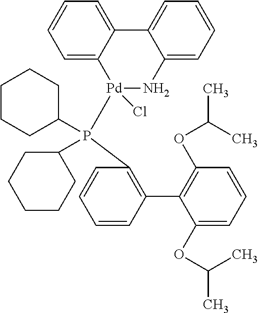 |
| SPhos Pd G2 | 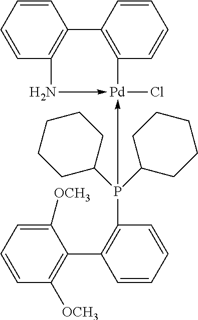 |
| t-AmOH | tert-Amyl alcohol |
| tBuXPhos Pd G3 | 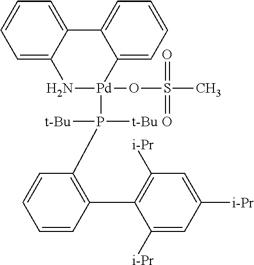 |
| THF | Tetrahydrofuran |
| TMG | Tetramethyl guanidine |
| TMSCN | Trimethylsilyl cyanide |
| XantPhos Pd G3 | 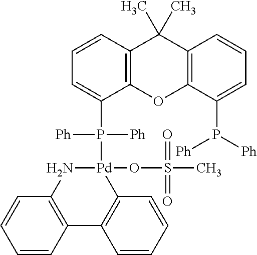 |
| Xphos Pd G2 | 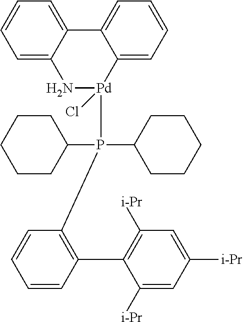 |
| Xphos Pd G3 | 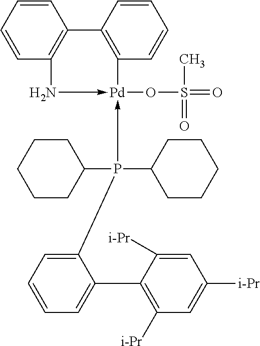 |
| Xphos Pd(crotyl)Cl | |

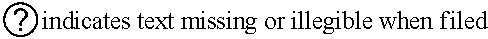

Example 1. Alternate Synthesis 1 of the lysine salt of Compound 8. The synthetic route of Alternate Synthesis 1 is outlined above in Scheme 2 and described in more detail below.

### Step 1:

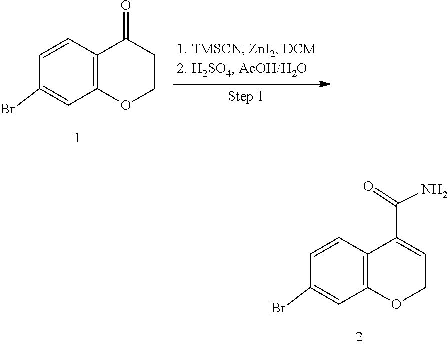 [SMILES: O=C1CCOc2cc(Br)ccc21 ; NC(=O)C1=CCOc2cc(Br)ccc21]

Compound 2 was prepared from Compound 1 using trimethylsilyl cyanide and ZnCl₂. More specifically, to a 500 L glass lined jacketed reactor under N₂ was charged 7-bromochroman-4-one (1) (7.7 kg), ZnI₂ (260 g), and dichloromethane (141 kg). TMSCN (5.1 kg) was then charged to the reactor, the solution was heated to reflux, and aged for approximately 3-5 hours whereupon it was assayed for conversion. The reaction mixture was then concentrated to 1-2 volume at <30° C. internal temperature with a stream of N₂. Glacial acetic acid (50.6 kg) was charged to the reaction mixture and the mixture was cooled to between 15-25° C. Water (5.0 kg) was charge to the mixture between 15-25° C., and then concentrated H₂SO₄ (38.2 kg) was added dropwise to the mixture keeping the internal temperature between 15-70° C. (actual ˜45° C.). The solution was then heated to 60-70° C. and aged (7-9 hr). The internal temperature of the mixture was adjusted to 50-60° C. and water (331 kg) was charged to the reactor dropwise in the same temperature range. The resulting suspension was stirred and aged (1-2 hr), then cooled to 5-15° C. at 10° C./hour, and the slurry was then filtered. The resulting wet cake was washed with water (144 kg) until the pH=6-7 and CN- was not detected, and subsequently dried. The crude product (18.3 kg) of a yellow solid was obtained. The resulting crude solid and ethyl acetate (302 kg) were then charged to a 500 L glass-lined jacketed reactor and subsequently aged (˜1 hr). To this mixture was then charged Ecosorb-941 activated carbon (800 g), the mixture was heated to 35-45° C., and aged (1-2 hr). The resulting slurry was cooled to 20-30° C. and filtered through Celite (6.0 kg). The Celite cake was washed twice with ethyl acetate (16 kg), the organic filtrates were combined, and concentrated to approximately 1 to 3 volumes under vacuum below 50° C. The resulting solution was solvent swapped with dichloromethane (138 kg) to generate a slurry via distillation crystallization <50° C. The resulting slurry was filtered, the wet cake washed with dichloromethane (5.0 kg), and subsequently dried at 45° C. for 8 hr yielding Compound 2 (6.08 kg) in 70% yield. ¹H NMR (500 MHz, CDCl₃) δ (ppm)=7.46 (d, J=8.2 Hz, 1H), 7.09 (dd, J=2.0, 8.2 Hz, 1H), 7.04 (d, J=2.0 Hz, 1H), 6.31 (t, J=3.9 Hz, 1H), 5.72 (br s, 2H), 4.81 (d, J=4.0 Hz, 2H). ¹³C NMR (126 MHz, CDCl₃) δ (ppm)=167.8, 154.9, 131.5, 126.8, 124.9, 124.5, 123.2, 119.8, 118.5, 77.2, 76.9, 76.7, 64.7. HRMS (ESI) m/z calculated for C₁₀H₉BrNO₂ (M+H) 253.9811; Found: 253.9817.

### Step 2:

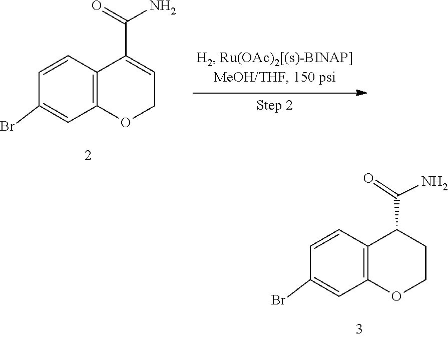 [SMILES: NC(=O)C1=CCOc2cc(Br)ccc21 ; NC(=O)[C@@H]1CCOc2cc(Br)ccc21]

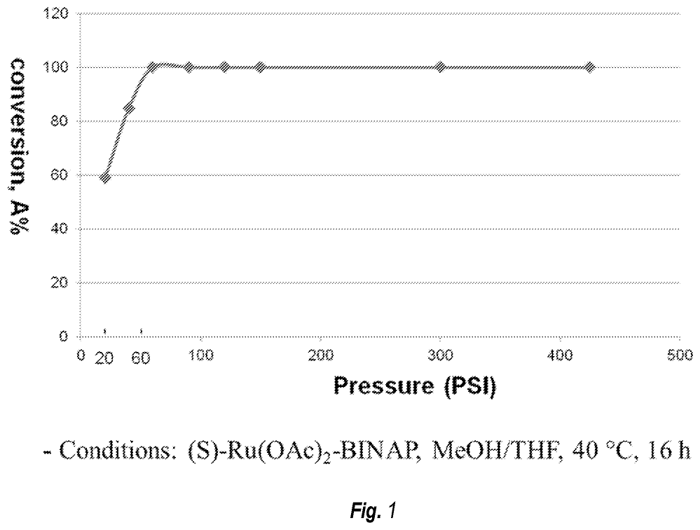

One issue with the hydrogenation of Compound 2 in the Discovery route (Scheme 1, above) was the high pressure (approximately 725 psi) and the lack of scalable isolation procedure. According, a pressure/temperature screen was done to evaluate the reaction. The results showed that there was little to no pressure or temperature effect on the selectivity of the hydrogenation. The results further showed that hydrogen pressure as low as 60 psi was sufficient to observe conversion to product. (See FIG. 1.) The final conditions were selected as the reaction conditions to balance equipment capability, reaction rate, and product quality.

Another problem with the hydrogenation step in the Discovery route (Scheme 1, above) was the isolation of Compound 3. Accordingly, an activated carbon screen was done to identify an effective medium for removing residual Ru. Based on these results, the scale-up synthesis of Compound 3 was conducted as follows.

To a 500 L stainless steel reactor under N₂ is charged with Compound 2 (5.35 kg), Ru(OAc)₂[(s)-BINAP](0.23 kg), THF (49 kg), and MeOH (44 kg). The contents were agitated and aged for (−0.5 hr) between 15-25° C. The reactor was then purged with N₂ three times and then H₂ three times, finally adjusted to 150 psi, heated (35-45° C.) and the mixture was aged (10-20 hr). Upon complete conversion, the temperature was adjusted (20-30° C.), activated carbon Excosorb C-941 (3.0 kg) was charged to the reactor and aged (16-24 h). The resulting slurry was filtered through Celite (5.0 kg) and pad was washed with THF (11.0 kg). The resulting combined organic filtrates were concentrated (6-7 volumes) below 50° C. under vacuum. The material was solvent switch to IPAc (114 kg) under vacuum <50° C. and assayed to reach <2% THF. The resulting IPAc solution was then heated (60-70° C.) and heptane (42.0 kg) was added dropwise (NLT 6 hr). The reaction was then aged (1-2 hr). The resulting slurry was cooled to 15-25° C. (6-10° C./h) and aged (5-10 hr). The resulting slurry was filtered and the cake washed with (1:2) IPAc/n-heptane (14.0 kg). The resulting wet cake was dried (40-50° C. for 24-36 hr) to yield the desired Compound 3 (4.8 kg, 89% yield, 95% ee). ¹H NMR (500 MHz, CD₃SOCD₃) δ (ppm)=7.61 (br s, 1H), 7.12-6.96 (m, 4H), 4.35-4.29 (m, 1H), 4.13 (ddd, J=3.5, 6.4, 10.5 Hz, 1H), 3.60 (t, J=5.8 Hz, 1H), 2.09-1.95 (m, 2H). ¹³C NMR (126 MHz, CD₃SOCD₃, 300 K) δ (ppm)=174.5, 155.5, 131.2, 122.8, 120.6, 119.7, 119.1, 63.7, 39.4, 25.0. HRMS (ESI) m/z calculated for C₁₀H₁₁BrNO₂ (M+H) 255.9968; Found: 255.9966.

### Steps 3 and 4:

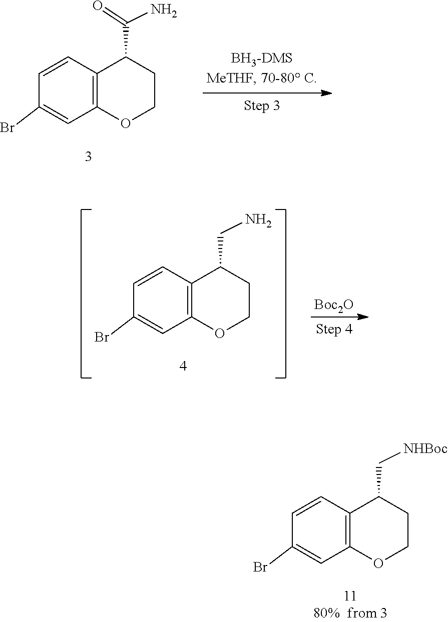

One issue with the amide reduction of Compound 3 in the Discovery route (Scheme 1, above) was that the procedure used excess BH₃·THF at 50-60° C. As discussed above, these conditions represent an explosion hazard. To avoid this hazard, an amide reduction reaction (discussed below) was developed using the thermally stable BH₃·DMS complex. The complex can be heated at higher temperature with significantly reduced risk for a runaway reaction.

Thus, the amide reduction of Compound 3 was conducted using the BH₃·DMS complex. Compound 4 was not isolated and instead was telescoped into Step 4, as shown in the scheme above.

While it was feasible to telescope the crude reaction mixture containing Compound 4 into protection Step 4, the subsequent crystallization of Compound 11 presented a significant challenge. Compound 11 has a tendency of oiling before forming a solid, leading to lower product quality and significant caking on the reactor walls. This behavior stems from a combination of several factors. Compound 11 has high solubility in a number of common organic solvents including heptane, limiting crystallization to alcohol/water mixtures (the solubility curve for DMSO/water is too steep). Compound 11 has a melting point of 65-70° C. and the melting point is depressed to 30° C. in n-PrOH/water mixtures (selected solvent system based on solubility). In addition, because the process was telescoped from Step 3, all the impurities from the amide reduction were carried into the crystallization.

The oiling challenge for Compound 11 was solved by incorporating a filtration of the crude reaction mixture after work-up through a plug of silica. This step likely removes polar impurities which contribute to the tendency of Compound 11 to oil. It was also discovered that slow addition of water and crystallization at temperatures below 20° C. were key to avoiding the oiling of Compound 11. The crystallization based on this protocol proved robust at scale-up was successfully implemented on an approximately 4 kg scale as shown in Table 1.

Thus, Compound 11 was prepared on large scale as follows. To a 250 L glass-lined vessel was charged Compound 3 (4.25 kg) and 2-MeTHF (57.0 kg) under N₂, and the solution was heated to 55-65° C. Upon reaching the temperature, neat BH₃·DMS (5.7 kg) was charged to the reaction mixture at 55-65° C. over approximately 1 hr. Once addition was completed, the mixture was heated (70-80° C.), aged (16-18 hr), and subsequently assayed via HPLC analysis. The mixture was then cooled (˜10-0° C.), and subsequent dropwise addition of 6N HCl (6.6 kg, charged over 3-4 hr) and water (7.0 kg, charged over 1-2 hr) yielded a quench solution (pH ˜1). A 5N NaOH solution (25.0 kg) was charged dropwise keeping the temperature below 10° C. adjusting the pH to 13-14. The temperature was then adjusted to 20-30° C., the layers separated, the aqueous layer was washed with 2-MeTHF (6 kg), and the resulting organic layers were combined. To the resulting organic layer was charged a solution of Boc₂O (4.35 kg, 1.05×wt.) in 2-MeTHF (4 kg) prepared in a separate reactor. The reactor train was rinsed with 2-MeTHF (3 kg) and this was added to the reaction mixture. The resulting mixture was allowed to age (14-16 hr). The reaction was then quenched and washed twice with 5 wt % NaCl solution (26 kg, 5.0×), filtered through a silica gel pad (3.0 kg, 0.5× wt.) and rinsed with 2-MeTHF (9 kg). The resulting solution was concentrated to 1-3 volume under vacuum <45° C. internal temperature. The solution was then solvent swapped with n-propyl alcohol (75.8 kg, 17.8× wt.) at or below 50° C. and then cooled to 20-30° C. internal temperature. The reaction mixture was then charged water (12.75 kg, 3.0× wt.) dropwise (over 1.5 hr), and seeded (130 g, 0.03× wt.). The temperature was adjusted to 0-15° C. (target 5° C. at a rate of 0.2° C./min.) and the slurry was aged (16-18 hr). Additional water (17.1 kg, 3.7× wt.) was charged dropwise (over ˜1.5 hr) to the slurry between 0-15° C. The mixture was then filtered between 0-10° C., and the resulting wet cake was washed with cold (1:4) n-propyl alcohol:water (15.0 kg, 3.5× wt.) to yield Compound 11 (4.65 kg, 92% ee). ¹H NMR (500 MHz, CD₃SOCD₃) δ (ppm)=7.11-7.00 (m, 3H), 6.95 (d, J=2.0 Hz, 1H), 4.17 (td, J=4.1, 11.0 Hz, 1H), 4.07 (dt, J=2.7, 10.6 Hz, 1H), 3.23 (td, J=5.4, 13.5 Hz, 1H), 3.03 (ddd, J=6.4, 9.5, 13.7 Hz, 1H), 2.81 (qd, J=4.7, 9.4 Hz, 1H), 1.94-1.75 (m, 2H), 1.38 (s, 9H). ¹³C NMR (126 MHz, CD₃SOCD₃) δ (ppm)=156.3, 156.0, 131.8, 123.6, 123.3, 119.9, 119.5, 78.2, 63.1, 45.2, 33.8, 28.7, 24.1. HRMS (ESI) m/z calculated for C₁₅H₁₉BrNO₃ (M−H) 340.0554; Found: 340.0538.

TABLE 1

| | | | | Wt/ | | Water | | |
|---|---|---|---|---|---|---|---|---|
| Input | Output | Yield | Purity | Wt | % | Content | ROI | Ru |
| (kg) | (kg) | (%) | (%) | (%) | ee | (%) | (%) | (ppm) |
| 4.2 | 4.65 | 80 | 99.5 | 98 | 92 | 0.3 | 0.1 | 123 |

### Step 5:

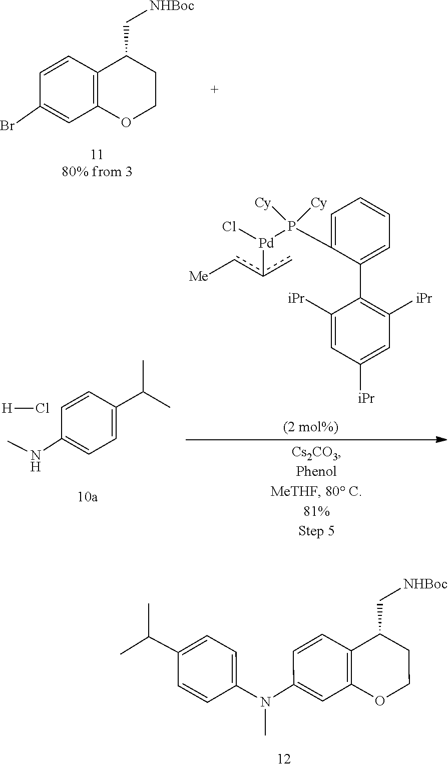

Compound 12 was synthesized from Compound 11 (prepared from steps 3 and 4, above) and Compound 10a.

Compound 10a can prepared from commercially available 4-isopropylaniline as follows. To a reactor is charged 4-isopropylaniline (500 g, 3.70 mol, 1.0 equiv.) and MeOH (2.5 L, 5× vol) under N₂. Paraformaldehyde (156 g, 5.20 mol, 1.4 equiv.) is added. The resultant slurry is stirred at 20° C. and 25 wt % solution of NaOMe (2.54 L, 3.0 equiv.) is charged, maintaining the internal temperature below 32° C. The resultant solution is allowed to stir at room temperature overnight (16 hr). To the solution is then charged NaBH₄ (182 g, 4.81 mol, 1.3 equiv.) portion-wise. The resultant solution is then heated at 60° C. for 2 hr. The reaction mixture is then cooled to room temperature and quenched with 1M KOH (2 L, 4× vol). The methanol is removed under reduced pressure. Water (2.5 L, 5× vol) and DCM (1.5 L, 3× vol) is added and the resultant emulsion is broken by filtration through a celite pad. The layers are separated, and the aqueous layer is further extracted with DCM (1 L, 2× vol.). The organic layer is dried over MgSO₄, filtered and concentrated to low volume under reduced pressure (˜2× vol). The mixture is placed in a reactor and cooled to −40° C. and 5M HCl in Et₂₀ (2.5 equiv.) is added over 30 mins and stirred for 1 hr. Petroleum ether (3 L, 6× vol) is then added and stirred for a further 1 hr. The precipitated solid is isolated by filtration and is washed with petroleum ether (2×1 L, 2× vol). The material is dried under vacuum to give crude Compound 10a in 95-110% yield at 80-88% purity. The crude product (400 g) is placed in a reactor and heptane (6 L, 15 vol) is added and heated to 90° C. To the slurry is added 1-butanol (0.6 L, 1.5 vol) over 25-30 mins until dissolution is obtained. The reaction is then heated for a further 60 mins. The solution is allowed to cool to room temperature over 3 hr and once at 30° C. the material is isolated by filtration and washed with heptane (2×1 L, 2.5 vol) and dried under vacuum to give 77-81% recovery at ˜97-98% purity by HPLC. A second recrystallisation is performed. Compound 10a (500 g) is placed in a reactor. Heptane (6.5 L, 13 vol) is added and heated to 90° C. and to the slurry was added 1-butanol (1.56-1.95 vol) over 25-30 mins until dissolution is obtained and then heated for a further 60 mins. The solution is allowed to cool to room temperature over 3 hr, filtered, and washed with heptane (2×1 L, 2.0 vol) to give Compound 10a (432 g). ¹H NMR (500 MHz, CD₃SOCD₃) δ (ppm)=7.16 (d, J=7.6 Hz, 2H), 7.09-6.93 (m, 3H), 6.40 (dd, J=2.4, 8.4 Hz, 1H), 6.23 (d, J=2.4 Hz, 1H), 4.13-3.97 (m, 2H), 3.36-3.27 (m, 1H), 3.24-3.12 (m, 3H), 3.07-2.93 (m, 1H), 2.88-2.70 (m, 2H), 1.95-1.75 (m, 2H), 1.39 (s, 9H), 1.19-1.18 (d, 6H). ¹³C NMR (126 MHz, CD₃SOCD₃) δ (ppm)=155.8, 154.9, 148.5, 146.3, 142.3, 129.6, 127.0, 122.1, 115.1, 110.8, 105.4, 77.6, 62.2, 45.0, 33.0, 32.7, 28.2, 28.1, 24.2, 24.0. HRMS (ESI) m/z calculated for C₂₅H₃₅N₂O₃ (M+H) 411.2642; Found: 411.2631.

The conversion of Compound 11 into Compound 12 via the C-N coupling reaction with Compound 10a was another challenge in the development of Alternate Synthesis 1. A series of studies were conducted to screen more than 48 different conditions to identify the optimal catalyst, solvent and base—see, e.g., Tables 2-4.

TABLE 2
Corrected DP:IS

| Catalyst | Toluene | DMAc | t-AmOH | 2-MeTHF |
|---|---|---|---|---|
| BrettPhos Pd G3 | 3.81 | 2.31 | 4.95 | 3.71 |
| CPhos Pd G3 | 2.71 | 3.93 | 5.54 | 2.40 |
| DavePhos Pd G3 | 1.04 | 2.93 | 3.06 | 0.92 |
| P(tBu)3 Pd G2 | 4.29 | 2.91 | 3.18 | 2.86 |
| JosiPhos Pd G3 | 1.73 | 0.32 | 0.97 | 0.29 |
| MorDalPhos Pd G3 | 0.23 | 0.89 | 1.76 | 0.82 |
| BINAP Pd G3 | 3.55 | 1.06 | 1.49 | 0.36 |
| RuPhos Pd G2 | 3.52 | 4.51 | 2.64 | 1.00 |
| SPhos Pd G2 | 4.41 | 3.80 | 5.05 | 3.49 |
| tBuXPhos Pd G3 | 0.70 | 0.26 | 1.65 | 1.98 |
| XantPhos Pd G3 | 2.03 | 2.65 | 0.28 | 1.42 |
| XPhos Pd G3 | 6.09 | 4.69 | 5.61 | 3.80 |

TABLE 3

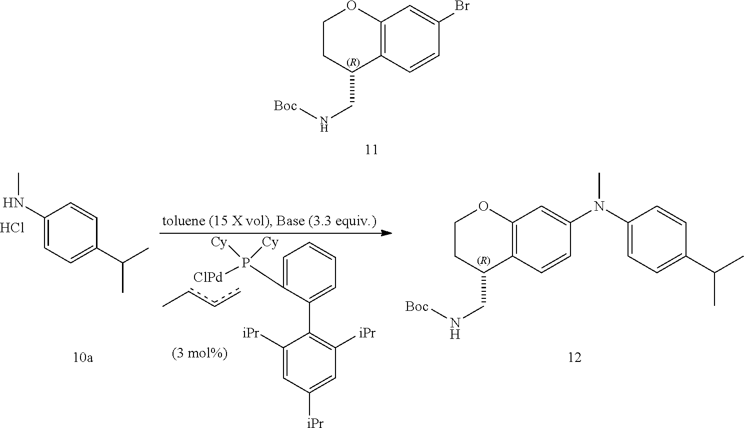

| Entry | Base | Water (equiv.) | Conv. (%) at 1.25 h | Conv. (%) at 15.25 h | Comment |
|---|---|---|---|---|---|
| 1 | Cs₂CO₃ | 3.0 | 11 | 100 | |
| 2 | Cs₂CO₃ | 3.0 | 8 | 100 | |
| 3 | Cs₂CO₃ | — | 2 | 61 | Biphasic (10X H₂O added) |
| 4 | Cs₂CO₃ | — | 81 | 100 | Added phenol (1.3 equiv) |
| 5 | NaOPh•3 H₂O | — | 4.5 | 33 | Biphasic/red aqueous |

TABLE 4

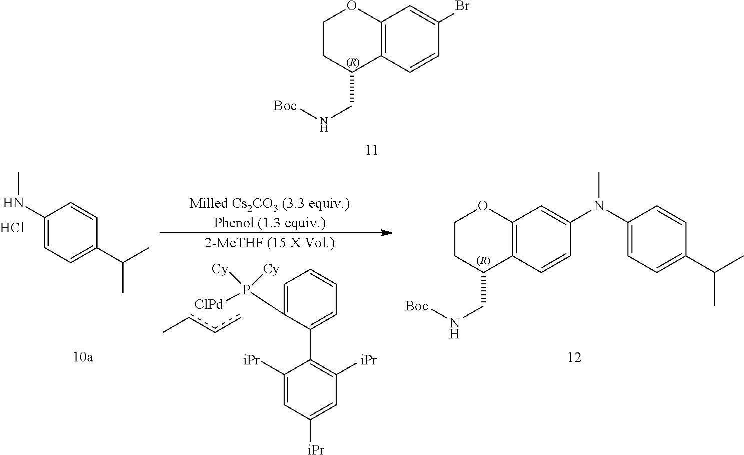

| Entry | Catalyst Charge (mol %) | Time (h) | Conv. (%) |
|---|---|---|---|
| 1 | 3 | 18 | 99.8 |
| 2 | 2 | 18 | 99.6 |
| 3 | 1 | 18 | 99.3 |
| 4 | 3 | 18.5 | 99.9 |
| 5 | 2 | 18.5 | 99.9 |
| 6 | 1 | 18.5 | 98.4 |

Table 2 shows the results of a catalyst screen using different solvents. DP:IS (desired product area to internal standard area) is a ratio term used to measure relative solution yields between a multi-well plate. The results give a relative rank of solution yields. While toluene gave good results under these conditions, ultimately it was not chosen because of poor solubility of the desired product.

The results in Table 3 show that the hydrated phenol base is not acceptable. The results also show the effects of added phenol compared to the addition of water. While phenol has been used previously to replace anhydrous KOPh and/or CsOPh (see Hartwig et. Al., J. Am. Chem. Soc. 2015, 137, 8460-8468), it had been used as a base rather than an additive. As such, the prior reactions using phenol were not executable on large scale.

Finally, Table 4 shows the acceptable catalyst loading range. The screening studies led to the discovery of significant catalyst activation with 1.3 equiv. of phenol. Reactions employing phenol proved to be robust and complete conversion was observed at scale from batch to batch. The increased catalyst activity under these conditions also allowed for the catalyst loading to be reduced to 1 or 2 mol %, as shown in Table 4.

The work-up and isolation of Compound 12 also presented a challenge. The work-up was hindered by emulsions which resulted in incomplete splits with significant product loss. It was observed that a black rag (presumably spend catalyst) was the source of the emulsion. The implementation of an in-process filtration through Celite prior to aqueous work-up addressed the emulsion issue and allowed for NaOH (aq) washes followed by water washes to remove all the inorganics and phenol.

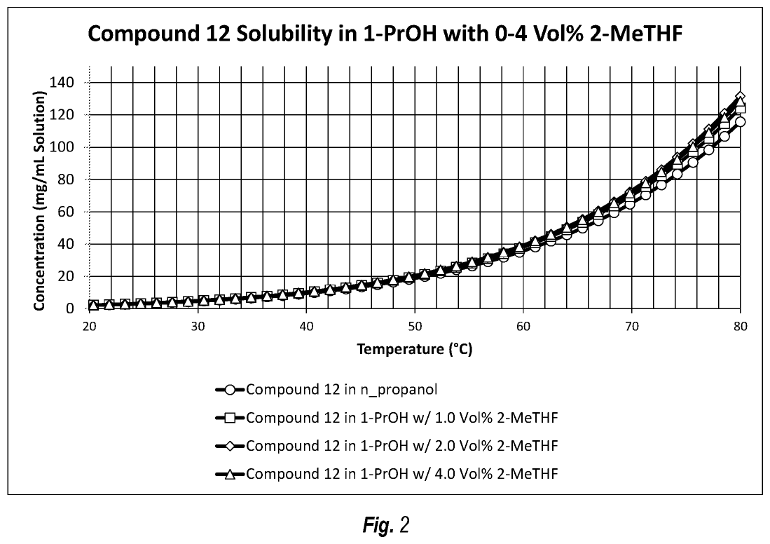

The crystallization of Compound 12 also required optimization. Compound 12 has low solubility in a number of common solvents, as shown in Table 5 below. Studies were undertaken to find the optimal solvent system of iPrOH/MeTHF. FIG. 2 shows the temperature dependent solubility curves of Compound 12 in 0 to 4 volume % 2-MeTHF in 1-propanol as measured in a Technobis Crystal-16 instrument.

TABLE 5

| Solvent | Solubility (mg/mL) |
|---|---|
| HOAC | 20 |
| MeOH | 3 |
| 1-PrOH | 5 |
| IPA | 3 |
| EtOAc | 22 |
| IPAc | 14 |
| MTBE | 11 |
| CPMe | 26 |
| toluene | 27 |
| heptane | 0 |
| THF | ≥150 |
| 2-MeTHF | 63 |
| MeCN | 5 |
| MEK | 28 |
| MIBK | 17 |
| DMAc | 77 |

In order to optimize chiral purity, an analysis of the filtrate of the final crystallization from Compound 12 was conducted (see Table 6). As can be seen in the table, as the wet cake is washed, the ee % of the filtrate increases, demonstrating that washing of the cake is helpful for obtaining high quality, high ee material. After ˜ the 3rd wash, the ee % of the filtrate can be observed to increase significantly from the 2nd.

TABLE 6

| | Compound 12 | Compound 12 | |
|---|---|---|---|
| Phase | Enantiomer 1 | Enantiomer 2 | ee |
| Filtrate | 15.41 | 16.1 | −2% |
| Wash 1 | 17.23 | 17.33 | 0% |
| Wash 2 | 24.5 | 16.16 | 21% |
| Wash 3 | 57.09 | 11.26 | 67% |

Based on the results in the various screening studies, the process to manufacture Compound 12 from Compound 11 was scaled up as follows. Compound 11 (3.00 kg, 1.0× wt.), phenol (1.073 kg, 0.358× wt., 1.3 equiv.), Cs₂CO₃ (9.416 kg, 3.139× wt., 3.3 equiv.), Compound 10a (1.790 kg, 0.597× wt., 1.1 equiv.) and palladium catalyst (0.118 kg, 0.039× wt., 0.02 equiv.) were charged in an appropriately sized reactor. The contents of the reactor were sparged with N₂, and then anhydrous 2-MeTHF (45 L, 15× vol.) was charged. The subsequent mixture was heated (75° C. +/−5° C.) and aged (6-16 hr) until complete conversion. The mixture was cooled (25° C.) and quenched by addition of water (0.3 L, 0.1× vol.) over 30 minutes. A suspension of celite (0.3 kg, 0.1× wt.) in 2-MeTHF (1.35 L, 0.45× vol.) was charged to the mixture, aged (˜10 min), and filtered. The reactor train and wet cake were rinsed with 2-MeTHF (3 L, LOX vol.). The organic filtrate and wash were combined and then heated to 30-35° C. The organic layer was extracted twice with 5M NaOH (12 L, 4× vol.), twice with water (12 L, 4× vol.), and the resulting organic layer was filtered through a polish filter. The extraction can alternatively be achieved using a less concentrated NaOH (e.g., 1M). The reaction mixture was dried via continuous distillation (Karl Fischer <1 wt. %) with 2-MeTHF and the reaction volume was reduced (˜15 L, 5× vol.). The mixture is heated (50-60° C.) and 1-propanol (6 L, 2× vol.) was added. The mixture was aged (˜1 hr) and then cooled (35-45° C.) where it was seeded (0.03×) and aged (2 hr). Additional 1-propanol (6 L, 2× vol) was slowly charged to the slurry, and the mixture was continuously distilled (5× vol. at 50-60° C.) with additional 1-propanol (until 2-MeTHF content was <1 vol %). The mixture was aged (NLT 60 mins) after distillation and then cooled (15-20° C.) and aged (NLT 6 hr). The resulting slurry was filtered, the wet cake was washed twice with 1-propanol (3 L, 1× vol.), 1-propanol (6 L, 2× vol.), and dried (40° C.) to yield Compound 12 (2.90 kg, >99.5%).

The results from three different batches are shown below in Table 7.

TABLE 7

| | | | | | | | Water | | |
|---|---|---|---|---|---|---|---|---|---|
| | Input | Output | Yield | Purity | Wt/Wt | | Content | | Pd/Ru |
| Batch | (kg) | (kg) | (%) | (%) | (%) | % ee | (%) | ROI | (ppm) |
| 1 | 0.198 | 0.195 | 79 | 99.3 | 102 | >99 | 0.1 | <0.1 | Pd: 462 |
| | | | | | | | | | Ru: 2 |
| 2 | 0.198 | 0.204 | 82 | 99.3 | 100 | >99 | 0.02 | <0.1 | Pd: 87 |
| | | | | | | | | | Ru: 2 |
| 3 | 3.0 | 2.9 | 81 | 99.3 | 98.4 | 99.5 | N/D | N/D | Pd: 263 |
| | | | | | | | | | Ru: 7 |

### Step 6

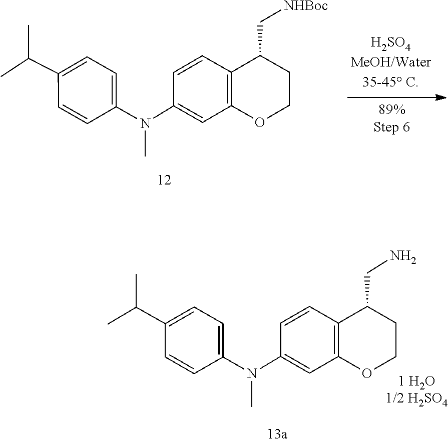

Compound 13a was prepared as follows. To an appropriately sized reactor was charged Compound 12 (2.90 kg, LOX wt.), methanol (20 L, 7× vol.), and H₂SO₄ (1.90 kg, 0.655× wt., 2.75 equiv.) via pump under N₂. Additional methanol (1 L, 0.345× vol.) was used to rinse the lines and wash the train. The mixture was heated (35-40° C.) and aged (˜6 hr) until reaction was complete. The reaction mixture was polish filtered, the train was washed with methanol (2.9 L, 1× vol.), and the filtrate and washes were combined. The resulting solution was heated (35-40° C.) water (11.6 L, 4× vol.) was added to the reaction mixture maintaining internal temperature (˜30 mins), and the mixture was aged (˜1 hr). A 6 wt % solution of aqueous sulfuric acid (11.6 L, 4× vol.) was charged to the reactor at a rate to maintain the temperature (1-2 hr). The mixture was then cooled (20-30° C.) over 1 hour and aged (1 hr). The resulting slurry was filtered, the wet cake and reactor train were twice rinse with 50% (v/v) aqueous methanol (5.8 L, 2× vol.), and the resulting wet cake was washed with water (5.8 L, 2× vol.). The resulting wet cake was dried (40-50° C.) to yield Compound 13a (2.36 kg). ¹H NMR (500 MHz, CD₃SOCD₃, 300 K) δ (ppm)=7.17 (br d, J=8.4 Hz, 2H), 7.10-6.88 (m, 3H), 6.40 (br dd, J=2.4, 8.5 Hz, 1H), 6.23 (br d, J=2.3 Hz, 1H), 4.14-3.97 (m, 2H), 3.21-3.09 (m, 3H), 3.00 (br d, J=8.7 Hz, 1H), 2.92-2.72 (m, 3H), 1.93 (br d, J=4.3 Hz, 2H), 1.19 (d, J=7.0 Hz, 6H). ¹³C NMR (126 MHz, CD₃SOCD₃) δ (ppm)=147.4, 141.9, 135.9, 120.7, 118.8, 118.6, 115.3, 114.4, 103.5, 102.2, 96.6, 54.1, 35.6, 31.2, 25.3, 23.5, 16.4, 15.0. HRMS (ESI) m/z calculated for C₂₀H₂₇N₂O (M+H) 311.2118; Found: 311.2112.

### Step 7:

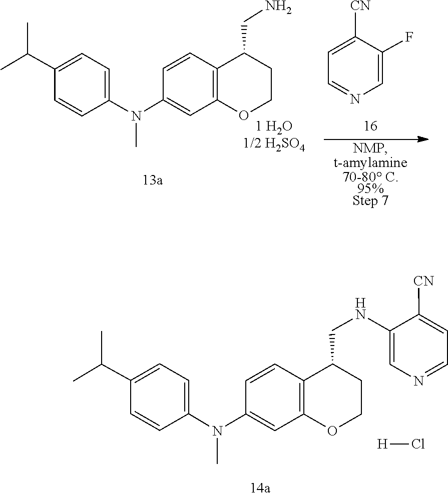

The next challenge in developing Alternate Synthesis 1 was the coupling to install the required pyridine moiety. A screening study was conducted using a number of starting materials and metal catalysts. For example, a catalyst screen was performed on 25 mg of racemic Compound A (see Table 9 below) using 12 different catalysts. ˜10 mole % catalyst was added with excess bromide substrate. After ˜14 hours at 70-75° C., three catalysts (Pd-173, 174 and 175) gave 20-25 Area % of racemic Compound C.

TABLE 9

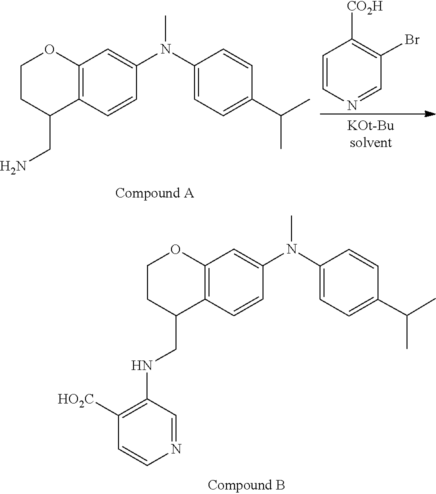

| Reference | | |
|---|---|---|
| No. | Description | Structure |
| Pd-173 | [BrettPhos Pd(crotyl)] OTf | 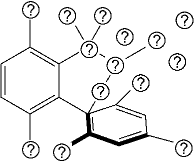 |
| Pd-174 | [tBuXPhos Pd(allyl)] OTf | 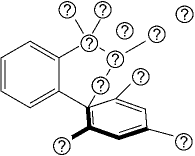 |
| Pd-175 | [tBuBrettPhos Pd(allyl)] OTf | 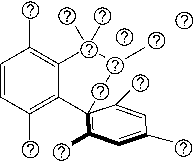 |

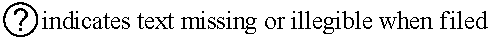

Further studies were conducted and summarized in Table 10, below. The preliminary data suggested that Pd-175 gave the highest conversion to the desired product (entries 1-3). The free base of the amine substrate showed higher conversion than the salts of amine (entries 3, 6, 10). Toluene as solvent gave better conversion than MeTHF and DMA (entries 3, 4 and 11). The addition of phenol had no improvement (entries 5 and 6). The reaction stalled after one hour and charging of more catalyst gave a bit higher conversion (entry 6). Less amount of catalyst reduced the conversion (entries 6 and 8). The iodide substrate gave lower conversion than the bromide one (entries 6 and 9) and ester substrate did not work well using Cs₂CO₃ base (entry 7).

TABLE 10

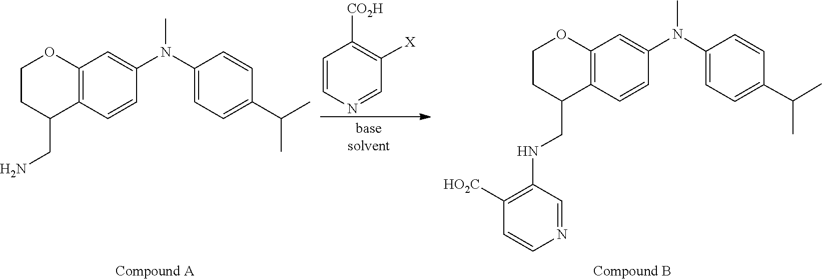

| | | | | | | Ratio of product B/starting |
|---|---|---|---|---|---|---|
| Entry | Amine substrate | Halide (X) | Catalyst | Solvent | Base | material A |
| 1 | PTSA salt | Br | Pd-173 | toluene | t-BuOK | 11/89 |
| 2 | PTSA salt | Br | Pd-174 | toluene | t-BuOK | 14/84 |
| 3 | PTSA salt | Br | Pd-175 | toluene | t-BuOK | 45/55 |
| 4 | PTSA salt | Br | Pd-175 | DMAC | t-BuOK | 31/69 |
| 5 | Free base | Br | Pd-175 | toluene | t-BuOK + | 57/43 |
| | | | | | phenol | |
| 6 | Free base | Br | Pd-175 | | t-BuOK | 71/29 (IPC one hour, 2 |
| | | | | | | hour) 82/18 (after reaction |
| | | | | | | stalled, more fresh catalyst |
| | | | | | | was added and continued |
| | | | | | | to monitor reaction) |
| 7 | Free base | X = Br, | Pd-175 | toluene | Cs₂CO₃ | NMT 2% desire product, |
| | | Ester | | | | major is SM |
| 8 | Free base | Br | Pd-175 | toluene | t-BuOK | 21/79 |
| 9 | Free base | I | Pd-175 | toluene | t-BuOK | 53/47 |
| 10 | H₂SO₄ salt | I | Pd-175 | toluene | t-BuOK | 47/53 |
| 11 | PTSA salt | Br | Pd-173 | MeTHF | t-BuOK | 25/75 |

PTSA = p-Toluenesulfonic acid

In addition to those screening studies, the possibility of using nucleophilic aromatic substitution (S_{N}Ar) was investigated. Another screening study was conducted (see Table 11 below) to identify an initial base and coupling partner (Compound 16a) with the appropriate leaving group (Cl or F). A previous screen (not shown) had identified that the best coupling partner was Compound 16a where X=F and DBU as the base.

TABLE 11

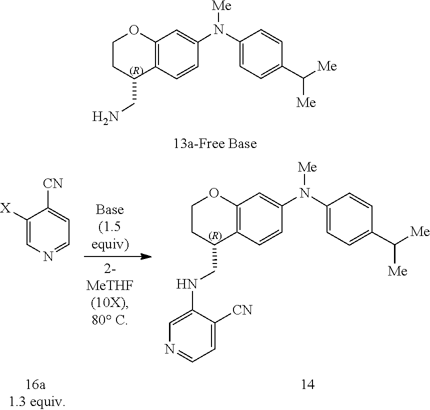

| Entry | Base | X | Conv. (%) | DP:IS |
|---|---|---|---|---|
| 1 | Cs₂CO₃ | F | 51 | 3.27 |
| 2 | pyridine | F | 54 | 3.33 |
| 3 | DBU | F | 94 | 6.03 |
| 4 | NMI | F | 76 | 2.89 |
| 5 | Et₃N | F | 43 | 2.66 |
| 6 | Dabco | F | 51 | 3.02 |
| 7 | Cs₂CO₃ | Cl | 0 | — |
| 8 | pyridine | Cl | 0 | — |
| 9 | DBU | Cl | 0 | — |
| 10 | NMI | Cl | 0 | — |
| 11 | Et₃N | Cl | 0 | — |
| 12 | Dabco | Cl | 0 | — |

Further screens were utilized to optimize both solvent and base utilizing Compound 16 (see Table 12, below). The purpose of the screen below was to define the optimal solvent and base to facilitate the S_{N}Ar reaction. Tert-amylamine provided the highest DP:IS ratios (desired product area to internal standard area) while the SM Pyr:IS ratio (starting material pyridine to internal standard) showed that the starting fluoropyridine remained at the end of these reaction conditions. DBU and TMG provided similar results which were secondary compared to tert-amylamine while also taking into consideration that the starting material, present in 50% excess, did not survive the reaction conditions. It was also observed that the addition of NMP as a co-solvent was using in the reaction.

TABLE 12

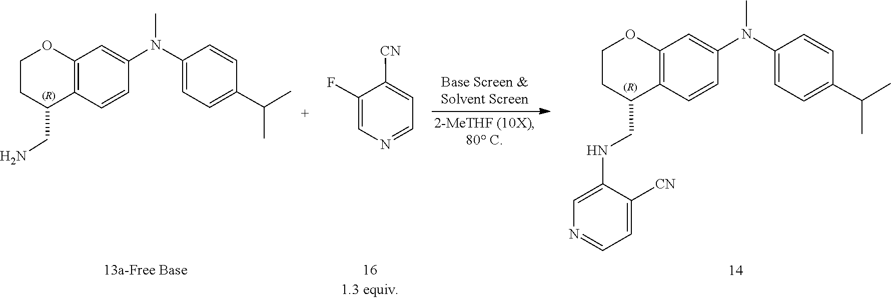

| Entry | Base | Solvent | Amount | Conversion (%) | DP:IS | SM Pyr:IS |
|---|---|---|---|---|---|---|
| 1 | 10 wt % Borate Buffer (aq) | 2-MeTHF | 10X Vol. | 74% | 2.464 | 0.467 |
| 2 | 10 wt % Borate Buffer (aq) | 2-MeTHF:NMP (9:1) | 10X Vol. | 87% | 2.534 | 0.132 |
| 3 | 10 wt % Borate Buffer (aq) | 2-MeTHF:NMP (7:3) | 10X Vol. | 87% | 1.473 | 0.188 |
| 4 | 20 wt % Borate Buffer (aq) | 2-MeTHF | 10X Vol. | 67% | 2.106 | 0.160 |
| 5 | 20 wt % Borate Buffer (aq) | 2-MeTHF:NMP (9:1) | 10X Vol. | 83% | 2.817 | 0.404 |
| 6 | 20 wt % Borate Buffer (aq) | 2-MeTHF:NMP (7:3) | 10X Vol. | 82% | 0.592 | 0.062 |
| 7 | 40 wt % Borate Buffer (aq) | 2-MeTHF | 10X Vol. | 64% | 2.053 | 0.528 |
| 8 | 40 wt % Borate Buffer (aq) | 2-MeTHF:NMP (9:1) | 10X Vol. | 44% | 1.215 | 0.405 |
| 9 | 40 wt % Borate Buffer (aq) | 2-MeTHF:NMP (7:3) | 10X Vol. | 65% | 1.168 | 0.307 |
| 10 | DBU | 2-MeTHF | 2 equiv. | 84% | 1.482 | 0.000 |
| 11 | DBU | 2-MeTHF:NMP (9:1) | 2 equiv. | 90% | 0.808 | 0.000 |
| 12 | DBU | 2-MeTHF:NMP (7:3) | 2 equiv. | 82% | 0.927 | 0.000 |
| 13 | tert-Amylamine | 2-MeTHF | 2 equiv. | 46% | 1.374 | 0.728 |
| 14 | tert-Amylamine | 2-MeTHF:NMP (9:1) | 2 equiv. | 91% | 3.049 | 0.307 |
| 15 | tert-Amylamine | 2-MeTHF:NMP (7:3) | 2 equiv. | 98% | 3.348 | 0.199 |
| 16 | TMG | 2-MeTHF | 2 equiv. | 66% | 1.919 | 0.000 |
| 17 | TMG | 2-MeTHF:NMP (9:1) | 2 equiv. | 98% | 1.401 | 0.000 |
| 18 | TMG | 2-MeTHF:NMP (7:3) | 2 equiv. | 92% | 1.482 | 0.000 |
| 19 | NaOTMS (1.0M) | 2-MeTHF | 2 equiv. | 0% | 0.000 | 0.007 |
| 20 | NaOTMS (1.0M) | 2-MeTHF:NMP (9:1) | 2 equiv. | 1% | 0.020 | 0.055 |
| 21 | NaOTMS (1.0M) | 2-MeTHF:NMP (7:3) | 2 equiv. | 4% | 0.063 | 0.000 |
| 22 | KHMDS (0.5M) | 2-MeTHF | 2 equiv. | 1% | 0.020 | 0.015 |
| 23 | KHMDS (0.5M) | 2-MeTHF:NMP (9:1) | 2 equiv. | 1% | 0.015 | 0.111 |
| 24 | KHMDS (0.5M) | 2-MeTHF:NMP (7:3) | 2 equiv. | 1% | 0.029 | 0.194 |

Accordingly, the large-scale synthesis of Compound 14a was conducted as follows. To an appropriately sized reactor was charged Compound 13a (2.30 kg, 1.0× wt.), Compound 16 (0.850 kg, 0.370× wt., 1.14 equiv.) and tert-amylamine (0.81 kg, 0.35× wt., 1.5 equiv.) under N₂. The reactor and contents were purged with N₂ and NMP (12.4 L, 5× vol.) was charged. The mixture was heated (70-75° C.) and aged (˜24 hr) until the reaction was completed. The reaction was then cooled (20-25° C.) and MTBE (23 L, 10× vol.) was charged. The reaction was then extracted with 5 wt % aqueous NaHCO₃ (11.5, 5× vol.), 5 wt % aqueous LiCl (11.5, 5× vol.), and three times with water (11.5, 5× vol.). Additional MTBE is charged to adjust the total volume (23 L, 10×). Isopropanol (11.5 L, 5× vol.) is charged to the reactor followed by the addition of freshly prepared 2M HCl in IPA (0.610 L, 0.265× vol.). The solution was then seeded (0.03×) to facilitate isolation of Compound 14a while maintaining the temperature (20-25° C.), although seeding is not necessary. The mixture was then aged (˜1 hr) and then freshly prepared 2M HCl in IPA (3.048 L, 1.325 L× vol) was charged over 5 hours maintaining the temperature (20-25° C.). The resulting orange suspension was aged (NLT 1 hr) and then filtered. The resulting wet cake was slurry washed twice with (2:1 v:v) MTBE/IPA (2.3 L, 1× vol.) and then displacement washed twice with 2:1 v:v MTBE/IPA (2.3 L, 1× vol.) and dried (40° C.), yielding Compound 14a (2.60 kg). ¹H NMR (500 MHz, CD₃SOCD₃) δ (ppm)=8.49 (s, 1H), 7.97 (d, J=5.3 Hz, 1H), 7.78 (br d, J=5.0 Hz, 1H), 7.27-7.09 (m, 4H), 6.97 (d, J=7.4 Hz, 2H), 6.40 (dd, J=2.4, 8.4 Hz, 1H), 6.26 (d, J=2.4 Hz, 1H), 4.18-4.08 (m, 2H), 3.65 (br dd, J=5.2, 13.7 Hz, 1H), 3.41 (br dd, J=10.2, 13.4 Hz, 1H), 3.25-3.03 (m, 4H), 2.85 (spt, J=6.9 Hz, 1H), 1.96-1.84 (m, 2H), 1.32-1.09 (m, 6H). ¹³C NMR (126 MHz, CD₃SOCD₃) δ (ppm)=155.0, 148.6, 146.2, 145.8, 142.4, 132.1, 131.5, 130.0, 128.0, 127.0, 122.1, 115.4, 114.5, 110.7, 105.4, 102.9, 62.2, 47.2, 32.7, 31.2, 24.2, 23.9. HRMS (ESI) m/z calculated for C₂₆H₂₉N₄O (M+H) 413.2336; Found: 413.2330.

The results from three different batches are reported in Table 13 below.

TABLE 13

| | | | | | | Water | Residual | |
|---|---|---|---|---|---|---|---|---|
| | Input | Output | Yield | Purity | Wt/Wt | Content | Solvents | Pd/Ru |
| Batch | (kg) | (kg) | (%) | (%) | (%) | (%) | (ppm) | (ppm) |
| 1 | 0.16 | 0.187 | 96 | 98.3 | 98.6 | 0.22 | MTBE: 5038 | Pd: 64 |
| | | | | | | | IPA: 4778 | Ru: <1 |
| 2 | 0.15 | 0.174 | 96 | 99.5 | 99.8 | 0.10 | MTBE: 4424 | Pd: 71 |
| | | | | | | | IPA: 4218 | Ru: <1 |
| 3 | 2.3 | 2.6 | 95.1 | 100 | 99.0 | N/D | MTBE: 6658 | Pd: 13 |
| | | | | | | | IPA: 4065 | Ru: 1 |

MBTE = methyl tert-butyl ether;
IPA = isopropyl alcohol

### Step 8:

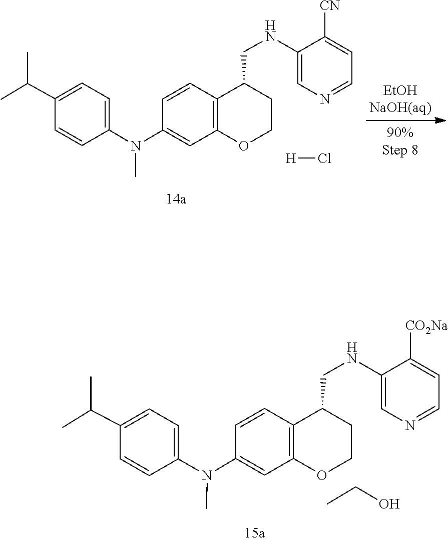

The next reaction in Alternate Synthesis 1 involved hydrolysis of the cyano group. The challenge was to develop a process that avoided the isolation of the free acid (Compound 8). The free acid tends to precipitate as an amorphous paste, making filtration at scale impossible, and its solubility is undetectable in water (low to neutral pH) and most common organic solvents. This was achieved by developing a process that affords the sodium salt EtOH solvate (Compound 15a). Compound 15a readily crystallizes from the hydrolysis conditions as a free-flowing solid that is easy to filter and dry. Compound 15a also provides a soluble intermediate for the synthesis of the lysine salt.

Compound 15a was prepared as follows. To an appropriately sized reactor was charged Compound 14a (500 g, 1.0× wt.) and 200 proof EtOH (2.5 L, 5× vol.). The reaction mixture was purged with N₂ and heated (50° C.). In a separate vessel, a solution consisting of 10 M NaOH (550 mL, 1.1× vol., 5.0 equiv.), water (250 mL, 0.5× vol.), and EtOH (500 mL, 1×) was prepared, and then charged to the reaction mixture via addition funnel (over 2.5 hr). Upon complete addition, the temperature was increased (70° C.) and the mixture was allowed to age (˜16 hr). Upon complete conversion, EtOH (4.25 L, 8.5× vol.) was slowly charged (2 hr) to the reaction mixture while maintaining the temperature (70° C.). After complete addition, the resulting slurry was cooled (3 hr) to ambient temperature (15-25° C.) and filtered. The wet cake was washed with EtOH (0.75 L, 1.5× vol.) three times and the material was dried in a vacuum oven (40° C.) to yield Compound 15a (514 g). ¹H NMR (500 MHz, CDCl₃) δ (ppm)=7.99 (br dd, J=5.0, 12.1 Hz, 1H), 7.69 (br s, 1H), 7.38 (br s, 1H), 7.25-7.11 (m, 1H), 7.08-6.93 (m, 2H), 6.91-6.78 (m, J=7.5 Hz, 2H), 6.72 (br s, 1H), 6.25 (br s, 1H), 6.20 (br s, 1H), 3.71 (ddd, J=2.6, 6.9, 14.0 Hz, 2H), 3.11 (br s, 1H), 3.01 (br s, 3H), 2.93-2.81 (m, 1H), 2.75 (td, J=6.6, 13.4 Hz, 4H), 1.78-1.49 (m, 2H), 1.28-1.17 (m, 1H), 1.13 (br d, J=6.7 Hz, 6H). ¹³C NMR (126 MHz, CD₃SOCD₃) δ (ppm)=164.6, 147.2, 141.2, 138.9, 137.1, 135.0, 126.6, 124.7, 121.1, 118.6, 117.8, 116.7, 114.3, 106.9, 102.9, 97.5, 54.6, 31.2, 25.3, 24.6, 17.3, 15.0. HRMS (ESI) m/z calculated for C₂₆H₃₀N₃O₃ (M+H) 432.2282; Found: 432.2273.

### Step 9:

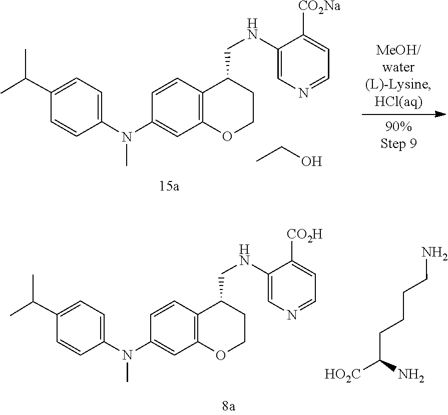

The final product (Compound 8a) was generated by reaction of Compound 15a with (L)-lysine under acidic conditions. More specifically, to an appropriately sized reactor was charged Compound 15a (559 g, 1.0 wt.) and L-lysine (540 g, 0.966× wt.). The reactor was then purged with N₂, and water (6.99 L, 12.5× vol.) was added. The resulting mixture was then headed (50° C.) and aged (˜1 hr). Methanol (2.52 L, 4.5× vol.) was charged in one portion and the reaction was then aged (10 mins). An aqueous solution of 2.12M HCl (671 mL, 1.2× vol.) was then charged (over 45 mins) by addition funnel, the mixture was cooled (40° C.), and Compound 8a seed (16 g, 0.03× wt.) was charged. The resulting slurry was aged (16 hr) and cooled to ambient temperature (15-25° C.). The solid was then isolated by filtration, the wet cake was washed by recycling the filtrate, and then the wet cake was washed three times with MeOH (1.12 L, 2× vol.). The resulting wet cake was dried in a vacuum oven (40-50° C.) resulting in Compound 8a (417 g). ¹H NMR (400 MHz, CD₃SOCD₃) δ (ppm)=6 ppm 9.20 (br s, 1H), 8.05 (s, 1H), 7.44-7.91 (m, 7H), 7.09-7.23 (m, 4H), 6.98 (d, J=8.44 Hz, 3H), 6.42 (dd, J=8.44, 2.32 Hz, 1H), 6.26 (d, J=2.32 Hz, 1H), 4.05-4.19 (m, 2H), 3.51 (br d, J=12.84 Hz, 1H), 3.12-3.25 (m, 5H), 2.71-3.03 (m, 5H), 1.81-2.03 (m, 3H), 1.28-1.77 (m, 8H), 1.20 (d, J=6.85 Hz, 8H). ¹³C NMR (100 MHz, CD₃SOCD₃) δ (ppm)=170.9, 170.2, 155.1, 148.5, 146.3, 144.9, 142.3, 135.5, 133.7, 129.7, 127.0, 125.2, 124.3, 122.0, 115.5, 110.9, 105.5, 62.5, 53.7, 47.3, 40.0 (only observed in DEPT) 38.3, 32.8, 32.1, 30.2, 26.7, 25.0, 24.0, 21.8. HRMS (ESI) m/z calculated for C₂₆H₃₀N₃O₃ (M+H) 432.2282; Found: 432.2290.

Example 2. Alternate Synthesis 2 of the lysine salt of Compound 8. The synthetic route of Alternate Synthesis 2 is outlined above in Scheme 4 and described in more detail below.

### Steps 1 and 2:

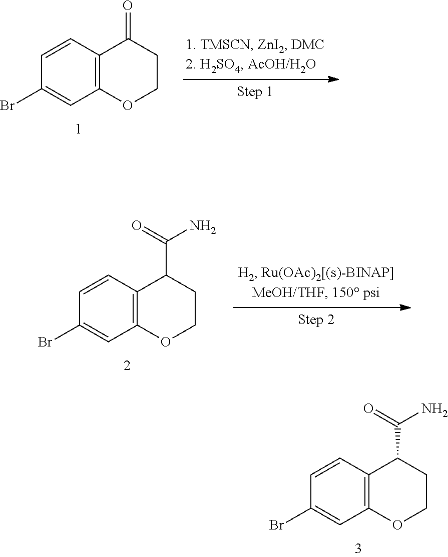 [SMILES: O=C1CCOc2cc(Br)ccc21 ; NC(=O)C1CCOc2cc(Br)ccc21 ; NC(=O)[C@@H]1CCOc2cc(Br)ccc21]

Compound 3 was prepared according to Steps 1 and 2 in Example 1 above.

### Step 3:

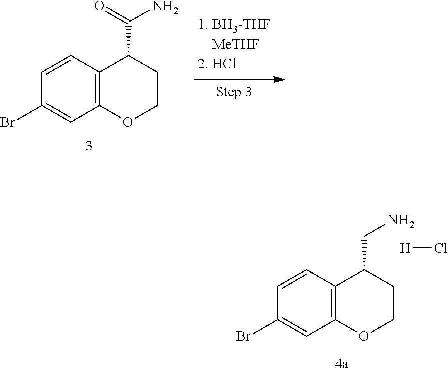

Compound 4a was prepared as follows. To an appropriately sized reactor was charged Compound 3 (1.0 g, 1.0× Wt) and MeTHF (16 mL) at 15-25° C., and borane-dimethyl sulfide (1.8 mL, 5.0 equiv.) was charged slowly. The mixture was then heated at 60-65° C. for at least 22 hours. After the reaction was complete, the mixture was cooled and quenched slowly with 6N aqueous HCl (1.5 mL, 1.5× Vol) and then 10 mL of water, maintaining the temperature below 20° C. The organic phase was separated and concentrated. Introduction of 3N HCl in CPME to the concentrate was followed by addition of isopropyl acetate (8 mL) to precipitate the product as an off-white solid. Filtration to isolate solid was followed by washing with isopropyl acetate. Drying under vacuum gave an off-white solid, Compound 4a (0.611 g, 61.2% yield, 99.4 Area % purity).

### Step 4:

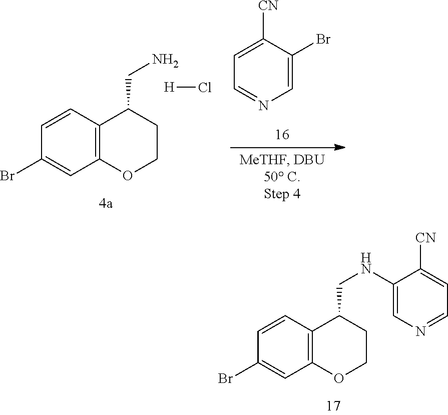

Compound 17 was prepared as follows. To a 100 mL reactor with N₂ inlet and pitched blade impeller was charged Compound 4a (5.62 g), Compound 16 (3.68 g) and 2-MeTHF (33 mL), and the mixture was inerted with N₂. DBU (7.52 mL) was charged into the mixture, and the mixture was heated (50° C.) and then aged (14.5 hr). Additional Compound 16 (1.41 g) was charged to the mixture, further aged (24 hr), and then cooled. The resulting slurry was filtered, the train and wet cake were washed with additional 2-MeTHF (20 mL) and then DCM (20 mL). The organic filtrates were combined and concentrated on a rotary evaporator yielding a brown liquid. The resulting crude product was purified via silica gel chromatography (120 g Isco column from Teledyne ISCO, loaded with DCM, and eluted with 10-70% EtOAc/hexanes). The resulting fractions were collected, concentrated and dried (40° C.) to yield Compound 17 as a light yellow solid (4.79 g, 69% yield).

### Step 5:

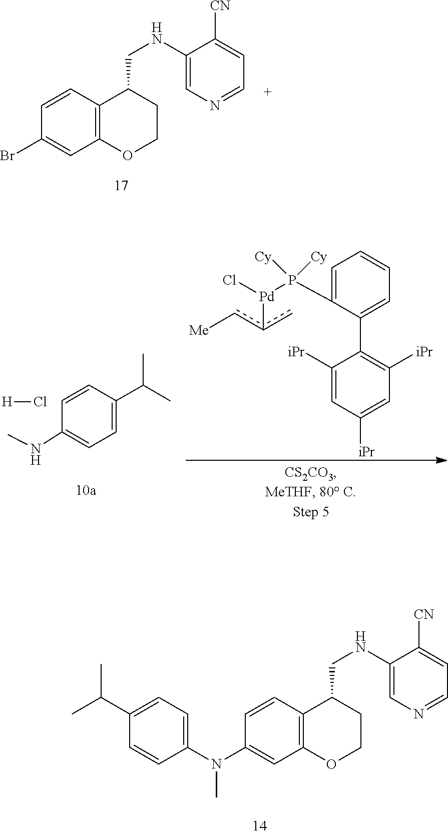

Compound 14 can be prepared from Compound 17 and Compound 10a according to the method described below, which was performed on the racemic mixture of Compound 17 (called Compound 17b herein) and yielded the racemic mixture of Compound 14 (called Compound 14b herein)).

To a 1000 mL 3-neck round bottom flask fitted with overhead stirrer was added Compound 17b (28.1 g), Compound 10a (16.74 g), Cs₂CO₃ (88.23 g), phenol (10.12 g) and the palladium catalyst (1.65 g). The reactor was flushed with N₂ for 25 min. Anhydrous, degassed 2-MeTHF (300 mL) was charged via cannula, the mixture was agitated, heated (75-80° C.), and aged (˜53 hr). The mixture was then cooled to room temperature and 2-MeTHF (50 mL) was charged. The mixture was then filtered. The reactor train and wet cake were washed with acetone (150 mL). The resulting organic layer was concentrated under reduced pressure to yield a black oil. Ethanol (35 mL) was charged with seeding of Compound 14b (50 mg) to facilitate isolation, and the mixture was allowed to stir overnight at room temperature. The resulting slurry was filtered, washed with MTBE (20 mL), and then dried to yield Compound 14b (26.5 g, 78% yield) as a yellow solid.

### Step 6:

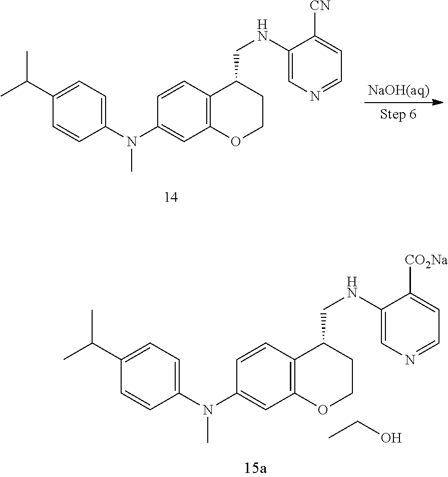

Compound 15a was prepared according to Step 8 in Example 1 above.

### Step 7:

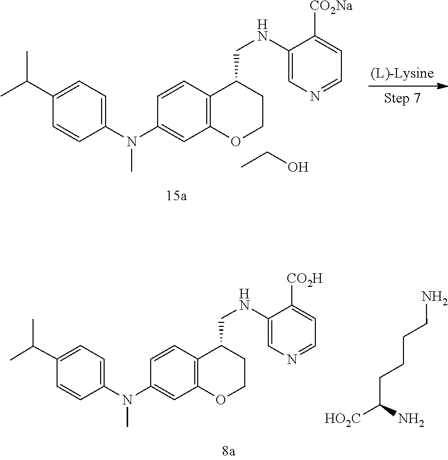

Compound 8a was prepared according to Step 9 in Example 1 above.

Although the present disclosure has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A method of preparing Compound 8: 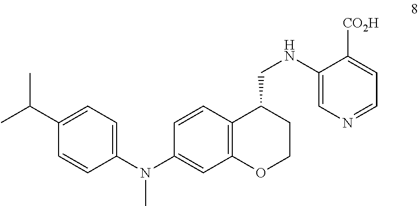 [SMILES: CC(C)c1ccc(N(C)c2ccc3c(c2)OCC[C@H]3CNc2cnccc2C(=O)O)cc1]
or a salt thereof, comprising the following steps:
(a) hydrolyzing Compound 14: 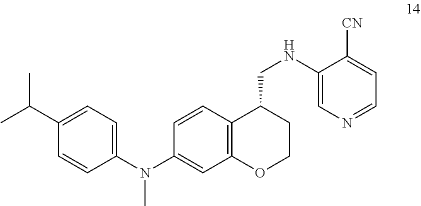 [SMILES: CC(C)c1ccc(N(C)c2ccc3c(c2)OCC[C@H]3CNc2cnccc2C#N)cc1]
or a salt thereof,
to form Compound 15: 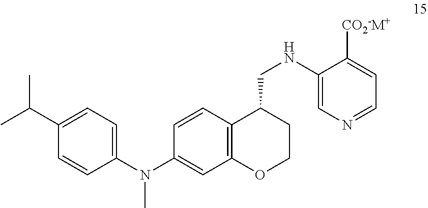
or a solvate thereof;
(b) reacting Compound 15, or a solvate thereof, with acid to form Compound 8; and
(c) optionally converting Compound 8 to a pharmaceutically acceptable salt thereof; and
wherein M⁺ is chosen from alkaline cations and protonated amine bases.

2. The method of claim 1, wherein M⁺ is chosen from Na⁺, K⁺, and a protonated dicyclohexylamine.

3. The method of claim 2, wherein M⁺ is Na⁺.

4. The method of claim 1, wherein step (a) is carried out on a salt of Compound 14.

5. The method of claim 4, wherein the salt is an HCl salt.

6. The method of claim 1, wherein step (a) is carried out using at least one base chosen from an alkaline hydroxide and a dicyclohexylamine.

7. The method of claim 6, wherein the alkaline hydroxide is chosen from NaOH and KOH.

8. The method of claim 7, wherein the alkaline hydroxide is NaOH.

9. The method of any one of claims 1-8, wherein step (a) is carried out using an alcohol as solvent.

10. The method of claim 9, wherein the alcohol is chosen from ethanol and methanol.

11. The method of claim 10, wherein the alcohol is ethanol.

12. The method of claim 11, wherein Compound 15 is formed as an ethanol solvate.

13. The method of any one of claims 1-12, wherein the acid in step (b) is HCl.

14. The method of any one of claims 1-13, wherein step (b) is carried out using aqueous alcohol as solvent.

15. The method of claim 14, wherein the alcohol is chosen from ethanol and methanol.

16. The method of claim 15, wherein the alcohol is methanol.

17. The method of any one of claims 1-16, wherein Compound 8 is reacted with lysine in step (c) to form a lysine salt 8a

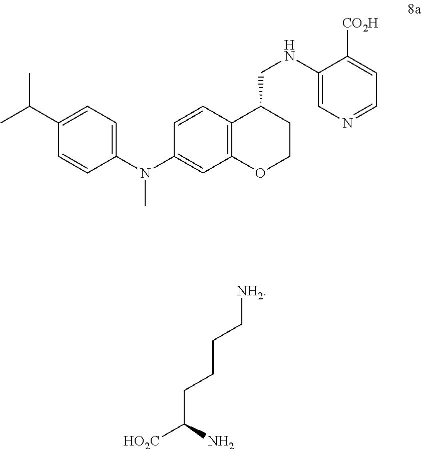 [SMILES: CC(C)c1ccc(N(C)c2ccc3c(c2)OCC[C@H]3CNc2cnccc2C(=O)O)cc1 ; NCCCC[C@@H](N)C(=O)O]

18. The method of any one of claims 1-17, wherein Compound 14, or a salt thereof, in step (a) is prepared by the following steps:
(i) reacting Compound 13: 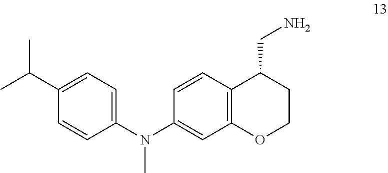 [SMILES: CC(C)c1ccc(N(C)c2ccc3c(c2)OCC[C@H]3CN)cc1]
or a salt and/or solvate thereof,
with Compound 16: 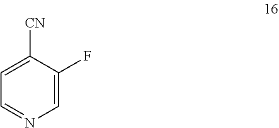
to form Compound 14; and
(ii) optionally converting Compound 14 into a salt thereof.

19. The method of claim 18, wherein step (i) is carried out using at least one polar aprotic solvent.

20. The method of claim 19, wherein the at least one polar aprotic solvent is N-methyl-2-pyrrolidone (NMP).

21. The method of any of claims 18-20, wherein step (i) is carried out using a base.

22. The method of claim 21, wherein the base is t-amylamine.

23. The method of any of claims 18-22, wherein step (i) is carried out at a temperature of about 60-100° C.

24. The method of claim 23, wherein step (i) is carried out at a temperature of about 70-80° C.

25. The method of any one of claims 18-24, wherein Compound 14 is reacted with HCl in step (ii) to form a hydrochloride salt.

26. The method of any one of claims 18-25, wherein Compound 13, or a salt and/or solvate thereof, is prepared by deprotecting Compound 12: 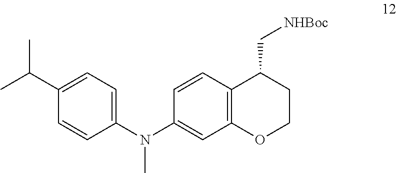 [SMILES: CC(C)c1ccc(N(C)c2ccc3c(c2)OCC[C@H]3CNC(=O)OC(C)(C)C)cc1]
under acidic conditions to form Compound 13 or salt and/or solvate thereof.

27. The method of claim 26, wherein the acidic conditions comprise H₂SO₄ in aqueous alcohol.

28. The method of claim 27, wherein the alcohol is methanol or ethanol.

29. The method of claim 28, wherein the alcohol is methanol.

30. The method of any one of claims 26-29, wherein deprotecting Compound 12 to form Compound 13, or a salt and/or solvate thereof, is carried out at a temperature of about 35-55° C.

31. The method of claim 30, wherein deprotecting Compound 12 to form Compound 13, or a salt and/or solvate thereof, is carried out at a temperature of about 35-45° C.

32. The method of any one of claims 26-31, wherein Compound 12 is prepared by reacting Compound 11: 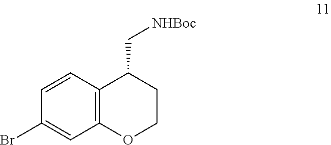 [SMILES: CC(C)(C)OC(=O)NC[C@@H]1CCOc2cc(Br)ccc21]
with Compound 10a: 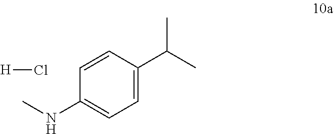
using a Palladium catalyst and phenol to form Compound 12.

33. The method of claim 32, wherein the Palladium catalyst is chosen from:

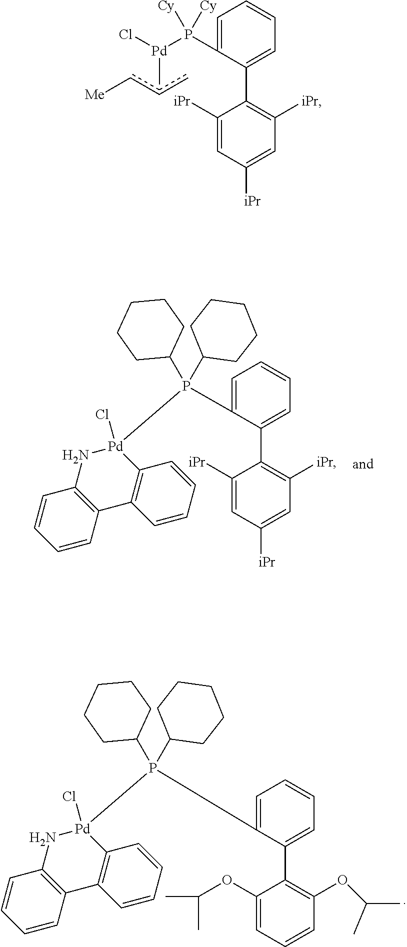

34. The method of claim 33, wherein the Palladium catalyst is:

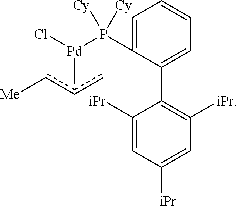

35. The method of claim 34, wherein the Palladium catalyst is used in an amount of about 2 mole %.

36. The method of any one of claims 32-35, wherein reacting Compound 11 with Compound 10a is carried out using 2-methyltetrahydrofuran as solvent.

37. The method of any one of claims 32-36, wherein reacting Compound 11 with Compound 10a is carried out at a temperature of about 80° C.

38. The method of any one of claims 32-37, wherein reacting Compound 11 with Compound 10a is carried out in the presence of a base.

39. The method of claim 38, wherein the base is Cs₂CO₃.

40. The method of any one of claims 32-39, wherein Compound 11 is prepared by reacting Compound 4: 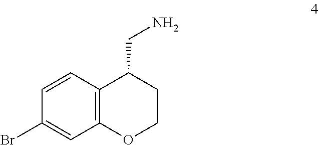 [SMILES: NC[C@@H]1CCOc2cc(Br)ccc21]
with Boc₂O to form Compound 11.

41. The method of claim 40, wherein Compound 4 is prepared by reacting Compound 3: 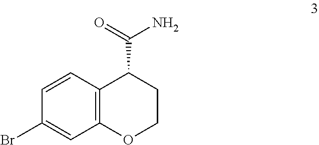 [SMILES: NC(=O)[C@@H]1CCOc2cc(Br)ccc21]
with BH₃·DMS to form Compound 4.

42. The method of claim 41, wherein reacting Compound 3 with BH₃·DMS is carried out using 2-methyltetrahydrofuran as solvent.

43. The method of claim 41 or 42, wherein reacting Compound 3 with BH₃·DMS is carried out at a temperature of about 70° C.

44. The method of any one of claims 1-17, wherein Compound 14, or a salt thereof, is prepared by reacting Compound 17: 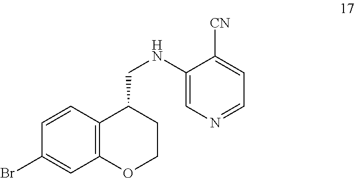 [SMILES: N#Cc1ccncc1NC[C@@H]1CCOc2cc(Br)ccc21]
with Compound 10a: 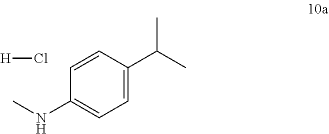
using a Palladium catalyst and phenol to form Compound 14, or a salt thereof.

45. The method of claim 44, wherein the Palladium catalyst is chosen from:

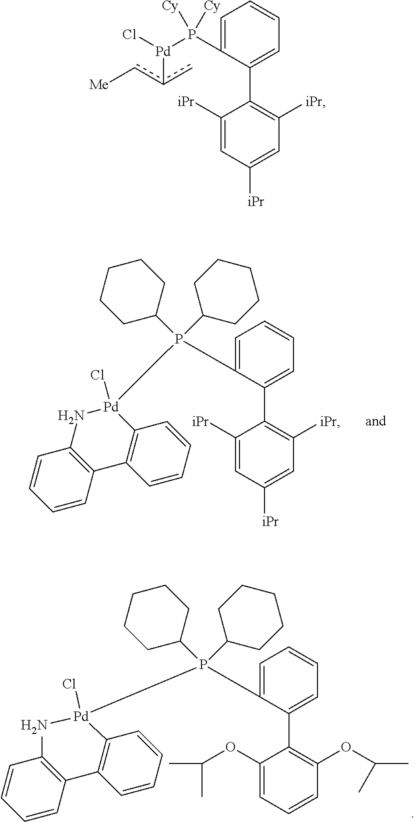

46. The method of claim 45, wherein the Palladium catalyst is:

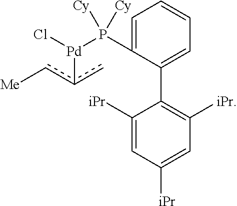

47. The method of any one of claims 44-46, wherein the Palladium catalyst is used in an amount of at least about 2 mole %.

48. The method of claim 47, wherein the Palladium catalyst is used in an amount of about 3 mole %.

49. The method of any one of claims 44-48, wherein reacting Compound 17 with Compound 10a is carried out using 2-methyltetrahydrofuran as solvent.

50. The method of any one of claims 44-49, wherein reacting Compound 17 with Compound 10a is carried out at a temperature of about 80° C.

51. The method of any one of claims 44-50, wherein reacting Compound 17 with Compound 10a is carried out in the presence of a base.

52. The method of claim 51, wherein the base is Cs₂CO₃.

53. The method of any one of claims 44-51, wherein Compound 17 is prepared by reacting Compound 4a: 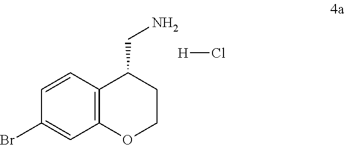
with Compound 16: 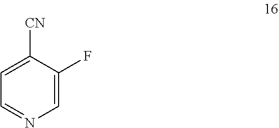
to form Compound 17.

54. The method of claim 53, wherein reacting Compound 4a with Compound 16 is carried out using 1,8-diazabicyclo[5.4.0]undec-7-ene (DBU) as a catalyst.

55. The method of claim 53 or 54, wherein reacting Compound 4a with Compound 16 is carried out using 2-methyltetrahydrofuran as solvent.

56. The method of any one of claims 53-55, wherein reacting Compound 4a with Compound 16 is carried out at a temperature of about 30-70° C.

57. The method of claim 56, wherein reacting Compound 4a with Compound 16 is carried out at a temperature of about 50° C.

58. The method of any one of claims 53-57, wherein Compound 4a is prepared by reacting Compound 3: 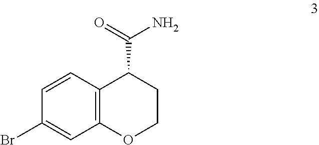 [SMILES: NC(=O)[C@@H]1CCOc2cc(Br)ccc21]
with BH₃·DMS and HCl to form Compound 4a.

59. The method of claim 58, wherein reacting Compound 3 with BH₃·DMS and HCl is carried out using 2-methyltetrahydrofuran as solvent.

60. The method of claim 58 or 59, wherein reacting Compound 3 with BH₃·DMS and HCl is carried out at a temperature of about 40-90° C.

61. The method of claim 60, wherein reacting Compound 3 with BH₃·DMS and HCl is carried out at a temperature of about 70° C.

62. The method of any one of claims 41-43 and 58-61, wherein Compound 3 is prepared by hydrogenating Compound 2: 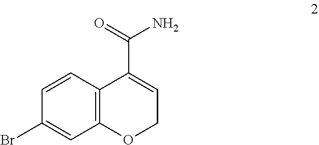 [SMILES: NC(=O)C1=CCOc2cc(Br)ccc21]
using a Ruthenium catalyst to form Compound 3.

63. The method of claim 62, wherein the Ruthenium catalyst is Ru(OAc)₂[(s)-BINAP].

64. The method of claim 62 or 63, wherein the hydrogenating is carried out using methanol, tetrahydrofuran, or a combination thereof as solvent.

65. The method of any one of claims 62-64, wherein the hydrogenating is carried out at a temperature of about 30-50° C.

66. The method of claim 65, wherein the hydrogenating is carried out at a temperature of about 35° C.

67. The method of any one of claims 62-66, wherein the hydrogenating is carried out under high pressure.

68. The method of claim 67, wherein the hydrogenating is carried out using a pressure of about 150 psi.

69. The method of any one of claims 62-68, wherein Compound 2 is prepared by reacting Compound 1: 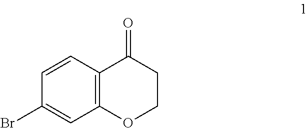 [SMILES: O=C1CCOc2cc(Br)ccc21]
with (i) trimethylsilyl cyanide and (ii) acid to form Compound 2.

70. The method of claim 69, wherein reacting Compound 1 with trimethylsilyl cyanide in (i) is carried out using ZnI₂.

71. The method of claim 69 or 70, wherein reacting Compound 1 with trimethylsilyl cyanide in (i) is carried out using dichloromethane as solvent.

72. The method of any one of claims 69-72, wherein the acid in (ii) is sulfuric acid, acetic acid, or a combination thereof.

73. The method of any one of claims 69-72, wherein reaction with acid in (ii) is carried out at a temperature of about 70-80° C.

74. A compound selected from: 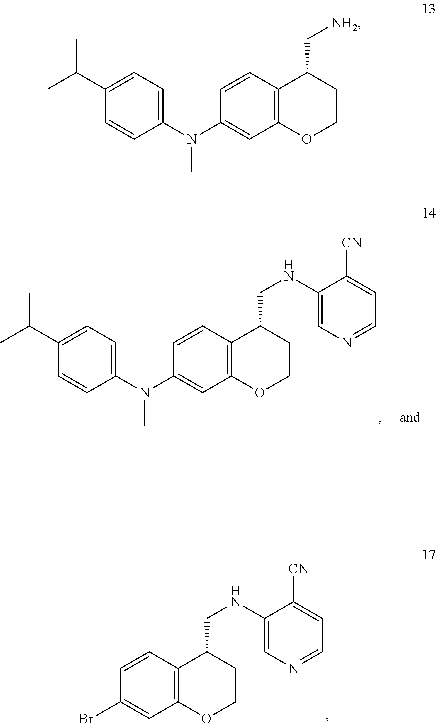 [SMILES: CC(C)c1ccc(N(C)c2ccc3c(c2)OCC[C@H]3CN)cc1 ; CC(C)c1ccc(N(C)c2ccc3c(c2)OCC[C@H]3CNc2cnccc2C#N)cc1 ; N#Cc1ccncc1NC[C@@H]1CCOc2cc(Br)ccc21]
or a salt and/or solvate thereof.

75. The compound of claim 74, which is:

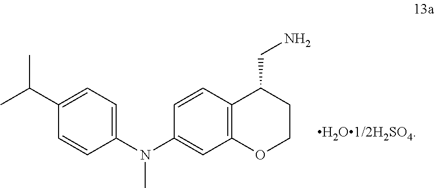 [SMILES: CC(C)c1ccc(N(C)c2ccc3c(c2)OCC[C@H]3CN)cc1]

76. The compound of claim 74, which is:

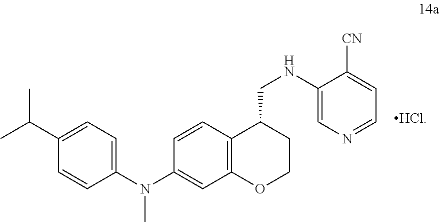

77. The compound of claim 74, which is: 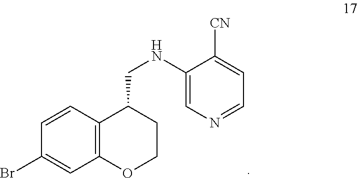 [SMILES: N#Cc1ccncc1NC[C@@H]1CCOc2cc(Br)ccc21]
